(19) United States
(12) Patent Application Publication
(10) Pub. No.: US 2025/0250234 A1
(43) Pub. Date: Aug. 7, 2025

(54) FLUOROALCOHOLS AS CO-SOLVENTS FOR CHEMICAL SYNTHESIS AND METHODS FOR PRODUCING THE SAME

(71) Applicant: University of Mississippi, University, MS (US)

(72) Inventors: David Colby, University, MS (US); Baharul Islam, University, MS (US)

(21) Appl. No.: 18/855,843

(22) PCT Filed: Apr. 11, 2023
(86) PCT No.: PCT/US2023/065613

§ 371 Date: Oct. 10, 2024

Related U.S. Application Data
(60) Provisional application No. 63/362,848, filed on Apr. 12, 2022.

(51) Int. Cl. C07D 213/61; C07C 45/67; C07C 201/12; C07D 317/54; C07D 333/56

(52) U.S. Cl. CPC C07D 213/61; C07C 45/673; C07C 201/12; C07D 317/54; C07D 333/56; C07B 2200/05

(57) ABSTRACT

In one aspect, the disclosure relates to a method for the in situ generation of formaldehyde for the hydroxymethylation of difluoroenolates to create 2,2-difluoroethanols having a range of solubilities, acidity, basicity, and other physical properties. The method also allows the production of formaldehyde-d₂ for hydroxy deuteromethylations and constitutes a new synthetic method for 2,2-difluoro-1,1-deuteroethanols. In one aspect, disclosed method is compatible with α,α-difluorobenzyl carbanions generated from the release of trifluoroacetate from electron-deficient aromatic and heteroaromatic rings and can be conducted under mild conditions with high yields. Also disclosed herein are 2,2-difluoroethanols generated by the disclosed method and methods of using the 2,2-difluoroethanols as solvents or co-solvents for organic synthesis, metal catalyzed reactions, asymmetric processes, carbohydrate chemistry, and medicinal chemistry.

### CROSS-REFERENCE TO RELATED APPLICATIONS

This application claims the benefit of U.S. Provisional Application No. 63/362,848, filed on Apr. 12, 2022, which is incorporated herein by reference in its entirety.

### STATEMENT REGARDING FEDERALLY SPONSORED RESEARCH OR DEVELOPMENT

This invention was made with government support under grant number R15DA046795, awarded by the National Institute on Drug Abuse of the National Institutes of Health and grant number P20GM130460, awarded by the National Institute of General Medical Sciences of the National Institutes of Health. The government has certain rights in the invention.

### BACKGROUND

Solvent effects are critical during the discovery process in order to produce an optimal yield of product as well as enable the discovery of new chemical transformations. Moreover, efficient synthetic strategies and solvents with low environmental impact are necessities in the pharmaceutical industry. Most chemical researchers do not design new classes of solvents in assist with reaction discovery and optimization, but instead rely on small group of commonly used solvents that are well characterized. Indeed, the production of any solvent requires a route must reliably make milliliter- or liter-quantities; however, the large-scale production of any organic molecule can be a considerable challenge, especially with structures with high degrees of complexity.

The fluorination of organic compounds is a powerful strategy to tune its physical properties through the unique electronegativity of the fluorine atom. The two currently known fluorinated solvents, trifluoroethanol and hexafluoroisopropanol, have found extensive use in many chemical reactions due to high levels of hydrogen-bond donation, low nucleophilicity, and high ionizing power. Moreover, trifluoroethanol has now been integrated into the multi-gram and multi-kilogram production of active pharmaceutical ingredients, which is a necessity for the healthcare field to treat diseases and conduct clinical trials. At the present, researchers will screen these two fluorinated alcohols to improve a reaction; however, in some cases, they both fail, and it is not clear why. Trifluoroethanol and hexafluoroisopropanol are achiral compounds and are limited in physical properties, thus also limiting their usefulness as solvents or co-solvents.

Although synthetic methods have been widely developed for the creation of fluorinated and trifluoromethylated structures, methods for many other classes of highly fluorinated structures are under-developed. This limitation has restricted the potential of using fluorinated alcohols to discover new reactions, because trifluoroethanol, hexafluoroisopropanol, as well as a few new fluorinated solvents, all display only trifluoromethyl groups.

Recent reports have demonstrated that using fluorinated alcohols as solvents promote state-of-the-art bond-activation and photoredox catalysis reactions whereas these processes fail when using the non-fluorinated counterparts. Fluorinated alcohols clearly enable many other chemical reactions such as C—C bond forming reactions, cyclizations, and solvolysis, but the mechanisms for this behavior are not well understood. The discovery of new classes of fluorinated alcohols to elucidate the mechanisms of the behavior and enable the design of new chemical reactions is needed to advance the field of organic synthesis and the production of pharmaceuticals.

Hydroxymethylation is a well-established synthetic process to generate valuable organic compounds. Notable examples have appeared recently in the total synthesis of natural products, drug discovery, and biosynthesis. The typical methods for hydroxymethylation require the use of formaldehyde or a reagent that serves as a formaldehyde equivalent. Formaldehyde is present in formalin or produced from trioxane or paraformaldehyde; other typical sources are 1-benzotriazole-1-methanol, N-(hydroxymethyl)phthalimide, and 1,3-oxathiolane-3,3-dioxide (FIG. 1). Alternatively, methods for the in situ generation of formaldehyde from dimethylsulfoxide (DMSO) or DMSO-like structures have been reported; however, they are not as convenient because excess heat is required and many side products are generated.

The incorporation of fluorine atoms on organic molecules is a common objective during drug development. Synthetic methods for the fluorination and trifluoromethylation of compounds are significantly more developed than strategies that create a difluoromethyl group. A mild process for the generation of α,α-difluoroenolates from the fragmentation of pentafluoro-gem-diols (FIG. 2) has been developed. Although these difluorinated intermediates react with aldehydes and imines, the compatibility of the transformation with formaldehyde has not been established. An expansion of the reactivity of formaldehyde to difluorocarbanions would potentially access to valuable 2,2-difluoroethanols and open new avenues for the synthesis of difluorinated targets.

Despite advances in synthetic research, there is still a scarcity of fluoroalcohols with wide-ranging and customizable physical properties that are useful as solvents or co-solvents for pharmaceuticals, fine chemicals, and other fluorochemicals. Ideally, new fluoroalcohols could be synthesized under mild conditions and with few or no undesirable side products. These needs and other needs are satisfied by the present disclosure.

### SUMMARY

In accordance with the purpose(s) of the present disclosure, as embodied and broadly described herein, the disclosure, in one aspect, relates to a method for the in situ generation of formaldehyde for the hydroxymethylation of difluoroenolates to create 2,2-difluoroethanols having a range of solubilities, acidity, basicity, and other physical properties. The method also allows the production of formaldehyde-d₂ for hydroxydeuteromethylations and constitutes a new synthetic method for 2,2-difluoro-1,1-deuteroethanols. In one aspect, disclosed method is compatible with α,α-difluorobenzyl carbanions generated from the release of trifluoroacetate from electron-deficient aromatic and heteroaromatic rings and can be conducted under mild conditions with high yields. Also disclosed herein are 2,2-difluoroethanols generated by the disclosed method and methods of using the 2,2-difluoroethanols as solvents or co-solvents for organic synthesis, metal catalyzed reactions, asymmetric processes, carbohydrate chemistry, and medicinal chemistry.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows common sources of formaldehyde.

FIG. 2 shows the generation of difluorocarbanions from the release of trifluoroacetate and the preparation of 2,2-difluoroethanols from the generation of formaldehyde in the presence of these reactive intermediates.

FIG. 3 is a scheme showing synthesis of difluoroethanols by hydroxymethylation from the in situ generation of formaldehyde and difluoroenolates, and synthesis of difluorodideuteroethanols by hydroxydeuteromethylation (percent deuterium incorporation is listed in brackets).

FIG. 4 is a scheme showing synthesis of difluoroethanols by hydroxymethylation from the in situ generation of formaldehyde and difluorobenzyl carbanions, and synthesis of difluorodideuteroethanols by hydroxydeuteromethylation (percent deuterium incorporation is listed in brackets).

FIG. 5 shows a proposed mechanism for the generation of formaldehyde from K₂CO₃, Br₂, and DMSO starting from the methyl(methylene)sulfonium cation. The changes in free energy and enthalpy for the fragmentation step are shown (in kcal/mol at 298.15 K). ¹H NMR data was obtained in acetone-de at 400 MHz at room temperature.

FIG. 6 shows conversion of 1,1,1,3,3-pentafluoro-3-(4-nitrophenyl)propane-2,2-diol into 1-(difluoromethyl)-4-nitrobenzene and 1-(deuterodifluoromethyl)-4-nitrobenzene with K₂CO₃ in DMSO at 65° C.

FIG. 7 shows free energies (G) and enthalpies (H) of three investigated processes (in kcal/mol at 298.15K).

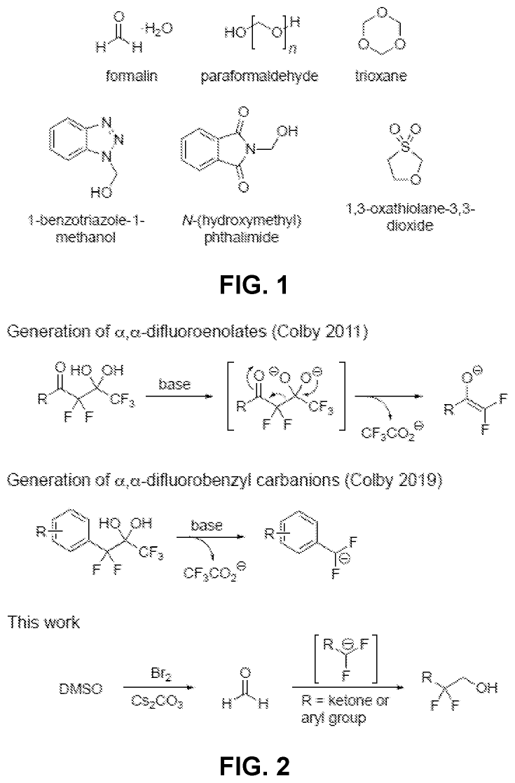

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Disclosed herein is a method for making a substituted 2,2-difluoroethanol, the method including at least the steps of contacting a pentafluoro-gem-diol of Formula I with molecular bromine, a carbonate base, and DMSO:

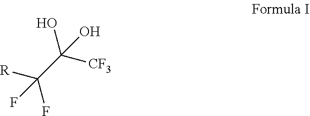

wherein R is a substituted or unsubstituted aryl or heteroaryl group having Formula II or a ketone having Formula III:

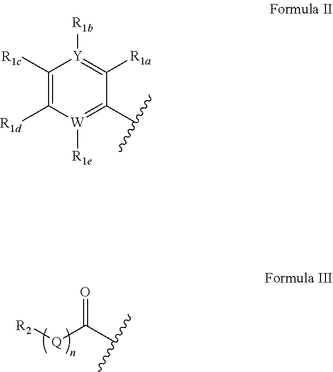

wherein, in Formula II, W and Y are independently C or N;
   wherein R₁ₐ, R_{1c}, and R_{1d} are independently selected from hydrogen, nitro, halogen, cyano, trifluoromethyl, hydroxyl, linear or branched C₁-C₁₀ alkyl, or any combination thereof;
   wherein, when Y is C, R_{1b} is selected from hydrogen, nitro, halogen, cyano, trifluoromethyl, hydroxyl, linear or branched C₁-C₁₀ alkyl, or any combination thereof; and
   wherein, when W is C, R₁ₑ is selected from hydrogen, nitro, halogen, cyano, trifluoromethyl, hydroxyl, linear or branched C₁-C₁₀ alkyl, or any combination thereof;
wherein, in Formula III, n is from 0 to 10;
   wherein when n is 1, Q is —CH₂—, and when n is from 2 to 10, Q is —CH₂— or —CH—; and
   wherein R₂ is selected from a substituted or unsubstituted C₆-C₁₀ aryl or heteroaryl group, an adamantyl group, or any combination thereof.

In one aspect, the pentafluoro-gem-diol of Formula I can be selected from:

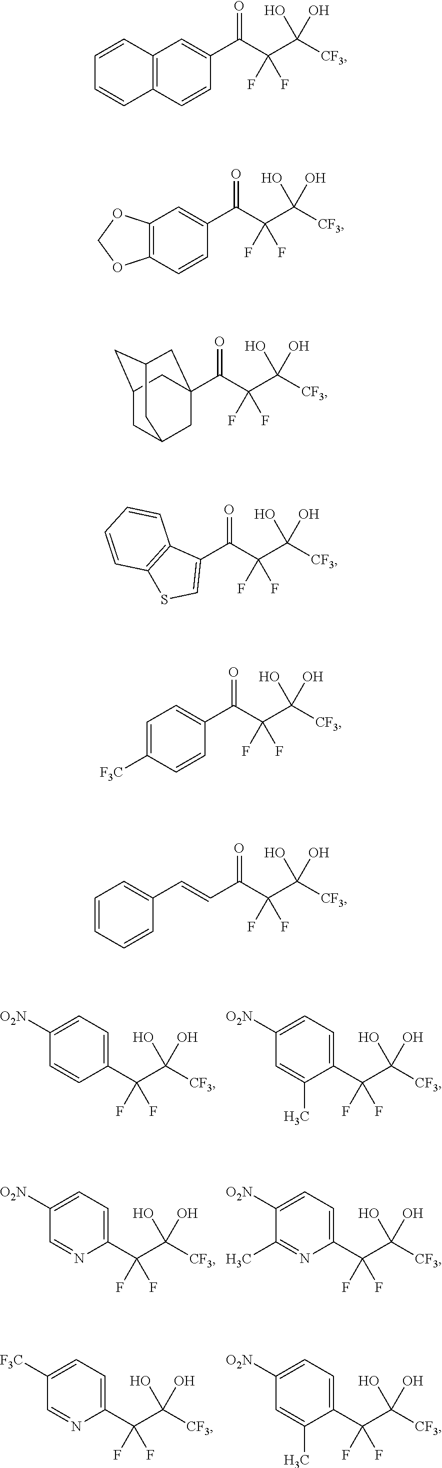

or any combination thereof.

In one aspect, the carbonate base can be Cs₂CO₃, Na₂CO₃, Li₂CO₃, or any combination thereof. In some aspects, the DMSO can be DMSO-d₆. In any of these aspects, the method can further include the step of contacting the pentafluoro-gem-diol with LiBr, molecular sieves, proton sponge, or any combination thereof.

In one aspect, the method can be performed at a temperature of form about 10° C. to about 60° C., or at about 10, 15, 20, 25, 30, 35, 40, 45, 50, 55, or about 60° C., or a combination of any of the foregoing values, or a range encompassing any of the foregoing values. In one aspect, the method can be performed under an inert atmosphere such as, for example, argon.

In another aspect, the substituted 2,2-difluoroethanol has Formula IV:

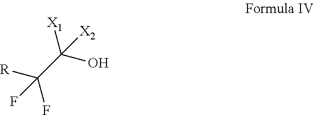

wherein X₁ and X₂ are H or D;
wherein R can be a substituted or unsubstituted aryl or heteroaryl group having Formula II or a ketone having Formula III:

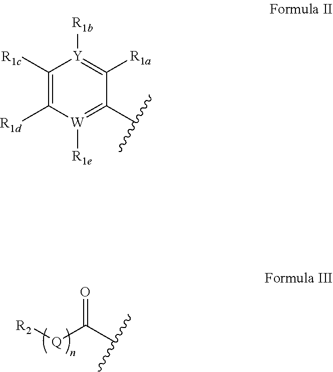

wherein, in Formula II, W and Y are independently C or N;
   wherein R₁ₐ, R_{1c}, and R₁ₐ are independently selected from hydrogen, nitro, halogen, cyano, trifluoromethyl, hydroxyl, linear or branched C₁-C₁₀ alkyl, or any combination thereof;
   wherein, when Y is C, R_{1b} is selected from hydrogen, nitro, halogen, cyano, trifluoromethyl, hydroxyl, linear or branched C₁-C₁₀ alkyl, or any combination thereof; and
   wherein, when W is C, R₁ₑ is selected from hydrogen, nitro, halogen, cyano, trifluoromethyl, hydroxyl, linear or branched C₁-C₁₀ alkyl, or any combination thereof;
wherein, in Formula III, n is from 0 to 10;
   wherein when n is 1, Q is —CH₂—, and when n is from 2 to 10, Q is —CH₂— or —CH—; and
   wherein R₂ is selected from a substituted or unsubstituted C₆-C₁₀ aryl or heteroaryl group, an adamantyl group, or any combination thereof.

In one aspect, X₁ and X₂ are D. In an alternative aspect, X₁ and X₂ are H. In still another aspect, R is Formula III and Q is 0. Further in this aspect, R₂ can be selected from:

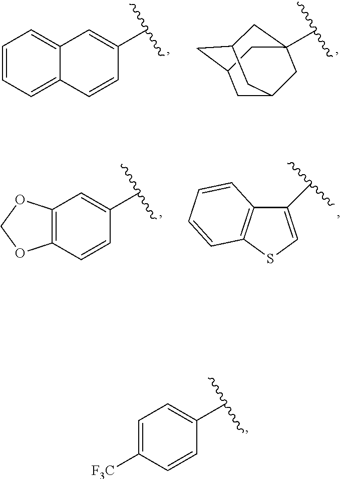 [SMILES: *c1ccc2ccccc2c1 ; *C12CC3CC(CC(C3)C1)C2 ; *c1ccc2c(c1)OCO2 ; *c1csc2ccccc12 ; *c1ccc(C(F)(F)F)cc1]

or any combination thereof.

In still another aspect, R can be Formula II. Further in this aspect, W can be N and Y can be C, or both Wand Y can be C.

In one aspect, the substituted 2,2-difluoroethanol of Formula IV is selected from:

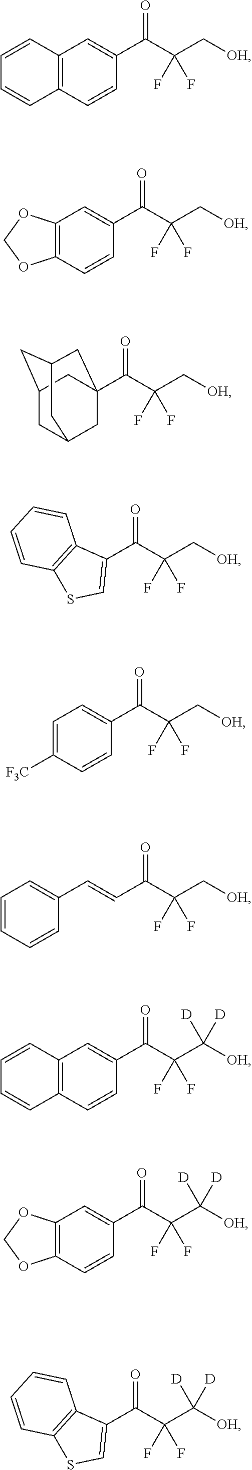

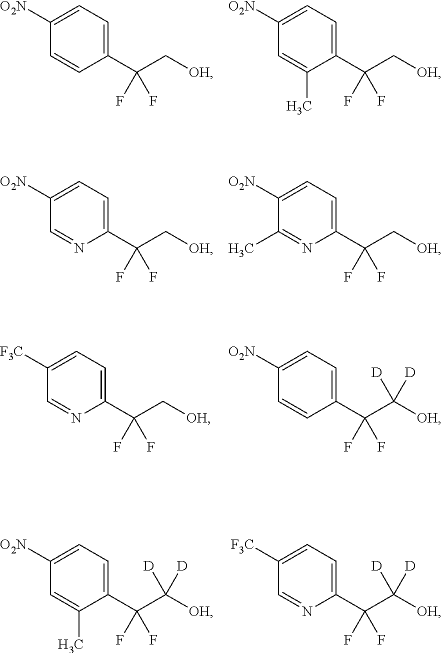

or any combination thereof.

In one aspect, the disclosed substituted 2,2-difluoroethanols are useful as solvents or co-solvents, or components of solvents or co-solvents, for organic synthesis. Further in this aspect, the solvents and co-solvents can be used to produce agrochemicals, polymers, petrochemicals, pharmaceuticals, and other fine chemicals, and can be used in the fabrication of electronic components including motherboards, microprocessors, and the like.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a carbonate base,” “a source of formaldehyde,” or “a difluoroenolate anion,” include, but are not limited to, mixtures or combinations of two or more such carbonate bases, sources of formaldehyde, or difluoroenolate anions, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “alkanediyl” as used herein, refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, and —CH₂CH₂CH₂— are non-limiting examples of alkanediyl groups.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹—OA² or —OA¹—(OA²)ₐ—OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) and —N(-alkyl)₂, where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula —(A¹O(O)C—A²—C(O)O)ₐ— or —(A¹O(O)C—A²—OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula —(A¹O—A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen” or “halide,” as used herein can be used interchangeably and refer to F, Cl, Br, or I.

The terms “pseudohalide,” “pseudohalogen” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3ring heteroatoms or wherein a pyridine ring is fused to a 5- or a 6-membered ring containing 1, 2or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxy” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” . . . “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{o}; —(CH₂)₀₋₄OR^{o}; —O(CH₂)₀₋₄R^{o}, —O—(CH₂)₀₋₄C(O)OR^{o}; —(CH₂)₀₋₄CH(OR^{o})₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —(CH₂)₀₋₄O(CH₂)₀₋₁-pyridyl which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR—, SC(S)SR^{o}; —(CH₂)₀₋₄SC(O)R^{o}; —(CH₂)₀₋₄C(O)NR^{o}₂; —C(S)NR^{o}₂; —C(S)SR^{o}; —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂NR^{o}₂; —N(R^{o})S(O)₂R^{o}; —N(OR^{o})R^{o}; —C(NH)NR^{o}₂; —P(O)₂R^{o}; —P(O)R^{o}₂; —OP(O)R^{o}₂; —OP(O)(OR^{o})₂; SiR^{o}₃; —(C₁₋₄ straight or branched alkylene)O—N(R^{o})₂; or —(C₁₋₄ straight or branched) alkylene)C(O)O—N(R^{o})₂, wherein each R^{o} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), are independently halogen, —(CH₂)₀₋₂R·, -(haloR·), —(CH₂)₀₋₂OH, —(CH₂)₀₋₂OR·, —(CH₂)₀₋₂CH(OR·)₂; —O(haloR·), —CN, —N₃, —(CH₂)₀₋₂C(O)R·, —(CH₂)₀₋₂C(O)OH, —(CH₂)₀₋₂C(O)OR·, —(CH₂)₀₋₂SR·, —(CH₂)₀₋₂SH, —(CH₂)₀₋₂NH₂, —(CH₂)₀₋₂NHR·, —(CH₂)₀₋₂NR·₂, —NO₂, —SiR·₃, —OSiR·₃, —C(O)SR·. —(C₁₋₄ straight or branched alkylene)C(O)OR·, or —SSR· wherein each R· is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═O and ═S.

Suitable divalent substituents on a saturated carbon atom of an “optionally substituted” group include the following: ═O, ═S, ═NNR*₂, ═NNHC(O)R*, ═NNHC(O)OR*; ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R·, -(haloR·), —OH, —OR·, —O(haloR·), —CN, —C(O)OH, —C(O)OR·, —NH₂, —NHR·, —NR·₂, or —NO₂, wherein each R· is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R·, -(haloR·), —OH, —OR·, —O(haloR·), —CN, —C(O)OH, —C(O)OR·, —NH₂, —NHR·, —NR·₂, or —NO₂, wherein each R· is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolyzable group” and “hydrolyzable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

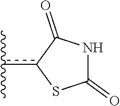

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example of an organic radical that comprises no inorganic atoms is a 5,6,7,8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, sulfur, fluorine and chlorine, such as ²H, ³H, ¹³C, ¹⁴C, ¹⁵N, ¹⁸O, ¹⁷O, ³⁵S, ¹⁸F, and ³⁶Cl, respectively. Compounds further comprise prodrugs thereof and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

It is also appreciated that certain compounds described herein can be present as an equilibrium of tautomers. For example, ketones with an α-hydrogen can exist in an equilibrium of the keto form and the enol form.

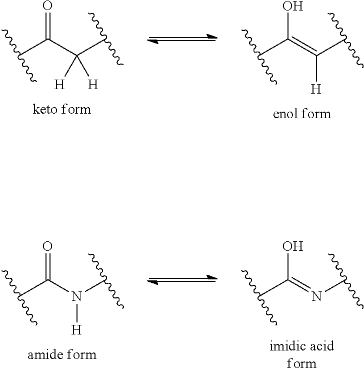 [SMILES: *CC(*)=O ; */C=C(/*)O ; *NC(*)=O ; */N=C(/*)O]

Likewise, amides with an N-hydrogen can exist in an equilibrium of the amide form and the imidic acid form. Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

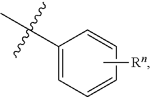

which is understood to be equivalent to a formula:

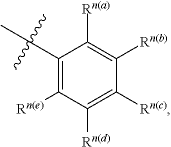

wherein n is typically an integer. That is, Rⁿ is understood to represent five independent substituents, R^{n(a)}, R^{n(b)}, R^{n(c)}, R^{n(d)}, and R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

A “solvent” as used herein refers to a substance that dissolves at least one or more solute, resulting in a solution. In one aspect, one or more solutes dissolved in a solvent can undergo a chemical reaction to produce a new substance. In a still further aspect, the pentafluoroisopropanols disclosed herein are useful as solvents in a variety of chemical processes as disclosed herein and may be particularly useful for reactions involving specific bond activation, photoredox catalysis, cyclization, solvolysis, and the like.

Meanwhile, a “co-solvent” as used herein refers to a substance added to a solvent in a small amount to increase the solubility of a poorly-soluble compound. In some aspects, co-solvents can be particularly useful to overcome hydrophobicity of poorly-soluble compounds, allowing their dissolution in aqueous solutions. In one aspect, the disclosed pentafluoroisopropanols are useful as co-solvents for producing and formulating pharmaceuticals, agrochemicals, petrochemicals, and the like.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

In various aspects, the disclosed compounds can possess at least one center of asymmetry, they can be present in the form of their racemates, in the form of the pure enantiomers and/or diastereomers or in the form of mixtures of these enantiomers and/or diastereomers. The stereoisomers can be present in the mixtures in any arbitrary proportions. In some aspects, provided this is possible, the disclosed compounds can be present in the form of the tautomers.

Thus, methods which are known per se can be used, for example, to separate the disclosed compounds which possess one or more chiral centers and occur as racemates into their optical isomers, i.e., enantiomers or diastereomers. The separation can be effected by means of column separation on chiral phases or by means of recrystallization from an optically active solvent or using an optically active acid or base or by means of derivatizing with an optically active reagent, such as an optically active alcohol, and subsequently cleaving off the residue.

Unless otherwise specified, pressures referred to herein are based on atmospheric pressure (i.e. one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### ASPECTS

The present disclosure can be described in accordance with the following numbered Aspects, which should not be confused with the claims.

Aspect 1. A method for making a substituted 2,2-difluoroethanol, the method comprising contacting a pentafluoro-gem-diol of Formula I with molecular bromine, a carbonate base, and DMSO:

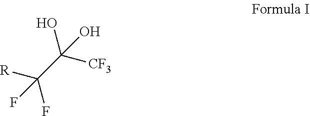

wherein R comprises a substituted or unsubstituted aryl or heteroaryl group having Formula II or a ketone having Formula III:

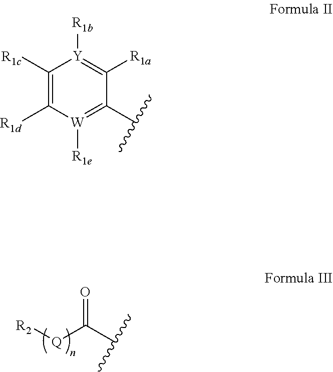

wherein, in Formula II, W and Y are independently C or N;
   wherein R₁ₐ, R_{1c}, and R_{1d} are independently selected from hydrogen, nitro, halogen, cyano, trifluoromethyl, hydroxyl, linear or branched C₁-C₁₀ alkyl, or any combination thereof;
   wherein, when Y is C, R_{1b} is selected from hydrogen, nitro, halogen, cyano, trifluoromethyl, hydroxyl, linear or branched C₁-C₁₀ alkyl, or any combination thereof; and
   wherein, when W is C, R₁ₑ is selected from hydrogen, nitro, halogen, cyano, trifluoromethyl, hydroxyl, linear or branched C₁-C₁₀ alkyl, or any combination thereof;
wherein, in Formula III, n is from 0 to 10;
   wherein when n is 1, Q is —CH₂—, and when n is from 2 to 10, Q is —CH₂— or —CH—; and
   wherein R₂ is selected from a substituted or unsubstituted C₆-C₁₀ aryl or heteroaryl group, an adamantyl group, or any combination thereof.

Aspect 2. The method of aspect 1, wherein the pentafluoro-gem-diol of Formula I is selected from:

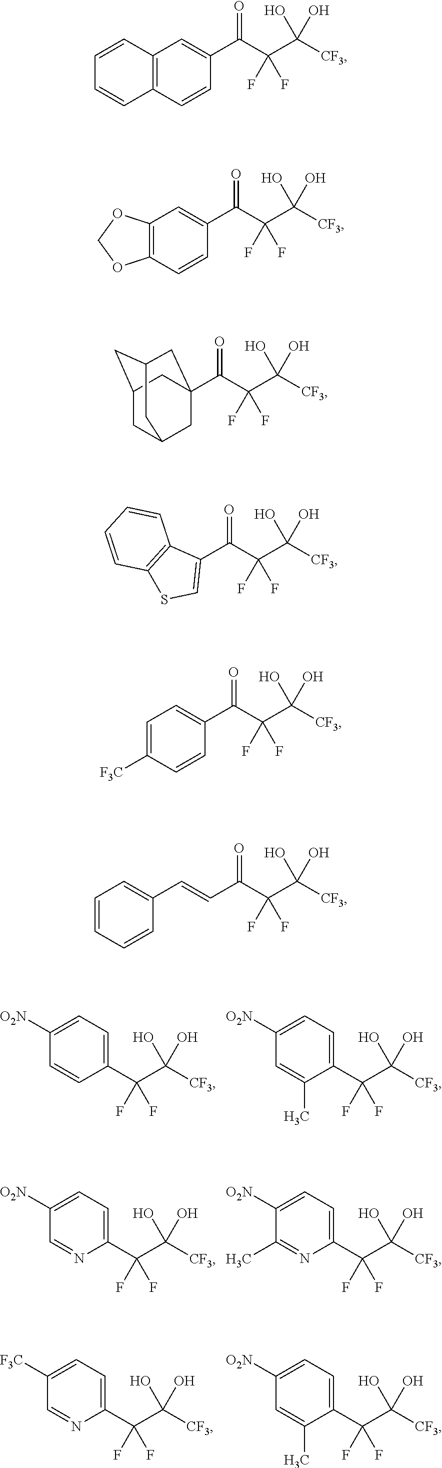

or any combination thereof.

Aspect 3. The method of aspect 1 or 2, wherein the carbonate base comprises Cs₂CO₃, Na₂CO₃, Li₂CO₃, or any combination thereof.

Aspect 4. The method of aspect 1 or 2, wherein the carbonate base is CS₂CO₃.

Aspect 5. The method of any one of the preceding aspects, wherein the DMSO is DMSO-d₆.

Aspect 6. The method of any one of the preceding aspects, further comprising contacting the pentafluoro-gem-diol with LiBr, molecular sieves, proton sponge, or any combination thereof.

Aspect 7. The method of any one of the preceding aspects, wherein the method is performed at a temperature of from about 10° C. to about 60° C.

Aspect 8. The method of any one of the preceding aspects, wherein the method is performed under argon.

Aspect 9. The method of any one of the preceding aspects, wherein the substituted 2,2-difluoroethanol has Formula IV:

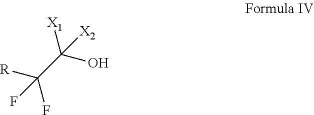

wherein X₁ and X₂ are H or D;
wherein R comprises a substituted or unsubstituted aryl or heteroaryl group having Formula II or a ketone having Formula III:

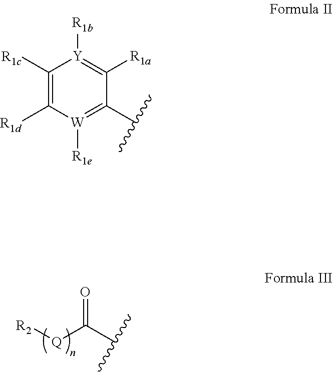

wherein, in Formula II, W and Y are independently C or N;
   wherein R₁ₐ, R_{1c}, and R_{1d} are independently selected from hydrogen, nitro, halogen, cyano, trifluoromethyl, hydroxyl, linear or branched C₁-C₁₀ alkyl, or any combination thereof;
   wherein, when Y is C, R_{1b} is selected from hydrogen, nitro, halogen, cyano, trifluoromethyl, hydroxyl, linear or branched C₁-C₁₀ alkyl, or any combination thereof; and
   wherein, when W is C, R₁ₑ is selected from hydrogen, nitro, halogen, cyano, trifluoromethyl, hydroxyl, linear or branched C₁-C₁₀ alkyl, or any combination thereof;
wherein, in Formula III, n is from 0 to 10;
   wherein when n is 1, Q is —CH₂—, and when n is from 2 to 10, Q is —CH₂— or —CH—; and
   wherein R₂ is selected from a substituted or unsubstituted C₆-C₁₀ aryl or heteroaryl group, an adamantyl group, or any combination thereof.

Aspect 10. The method of aspect 9, wherein the substituted 2,2-difluoroethanol of Formula IV is selected from:

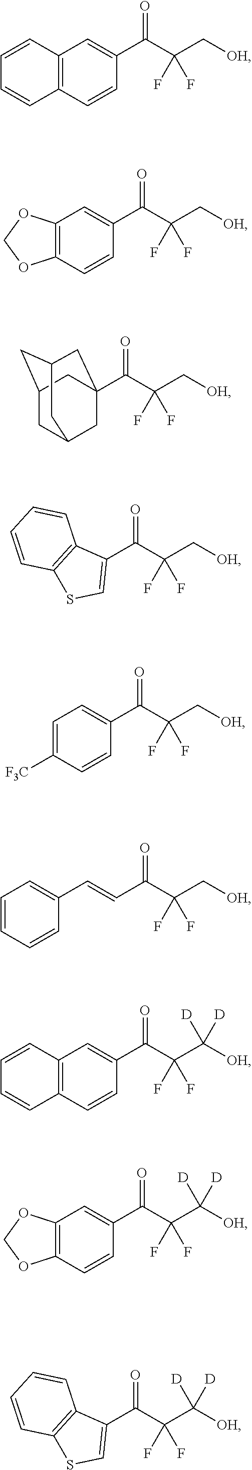

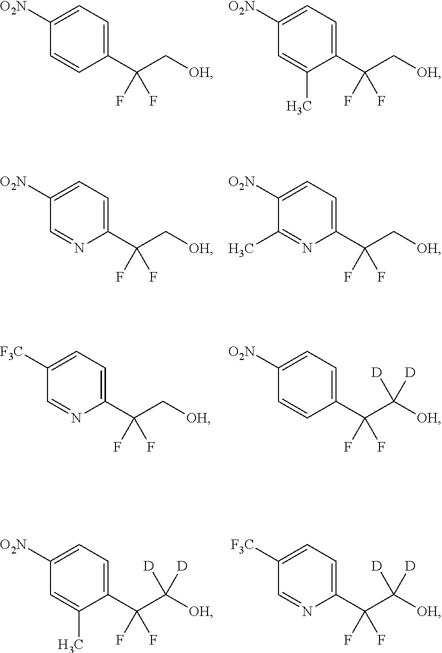

or any combination thereof.

Aspect 11. A substituted 2,2-difluoroethanol having Formula IV:

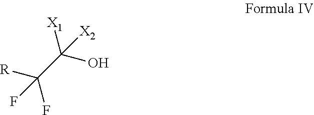

wherein X₁ and X₂ are H or D;
wherein R comprises a substituted or unsubstituted aryl or heteroaryl group having Formula II or a ketone having Formula III:

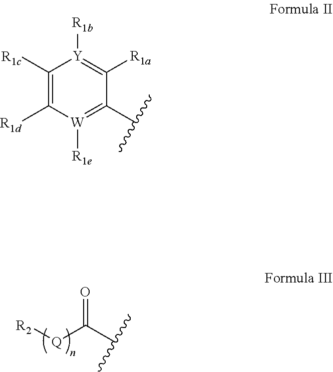

wherein, in Formula II, W and Y are independently C or N;
   wherein R₁ₐ, R_{1c}, and R_{1d} are independently selected from hydrogen, nitro, halogen, cyano, trifluoromethyl, hydroxyl, linear or branched C₁-C₁₀ alkyl, or any combination thereof;
   wherein, when Y is C, R_{1b} is selected from hydrogen, nitro, halogen, cyano, trifluoromethyl, hydroxyl, linear or branched C₁-C₁₀ alkyl, or any combination thereof; and
   wherein, when W is C, R₁ₑ is selected from hydrogen, nitro, halogen, cyano, trifluoromethyl, hydroxyl, linear or branched C₁-C₁₀ alkyl, or any combination thereof;
wherein, in Formula III, n is from 0 to 10;
   wherein when n is 1, Q is —CH₂—, and when n is from 2 to 10, Q is —CH₂— or —CH—; and
   wherein R₂ is selected from a substituted or unsubstituted C₆-C₁₀ aryl or heteroaryl group, an adamantyl group, or any combination thereof.

Aspect 12. The substituted 2,2-difluoroethanol of aspect 11, wherein X₁ and X₂ are D.

Aspect 13. The substituted 2,2-difluoroethanol of aspect 11, wherein X₁ and X₂ are H.

Aspect 14. The substituted 2,2-difluoroethanol of any one of aspects 11-13, wherein R is Formula III and Q is 0.

Aspect 15. The substituted 2,2-difluoroethanol of aspect 14, wherein R₂ is selected from

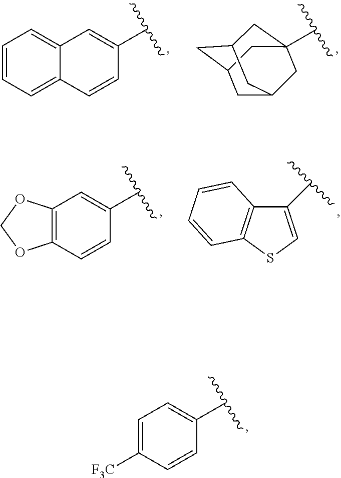 [SMILES: *c1ccc2ccccc2c1 ; *C12CC3CC(CC(C3)C1)C2 ; *c1ccc2c(c1)OCO2 ; *c1csc2ccccc12 ; *c1ccc(C(F)(F)F)cc1]

or any combination thereof.

Aspect 16. The substituted 2,2-difluoroethanol of any one of aspects 11-13, wherein R is Formula II.

Aspect 17. The substituted 2,2-difluoroethanol of aspect 16, wherein W is N and Y is C.

Aspect 18. The substituted 2,2-difluoroethanol of aspect 16, wherein W and Y are both C.

Aspect 19. The substituted 2,2-difluoroethanol of any one of aspects 11-18, wherein the substituted 2,2-difluoroethanol of Formula IV is selected from:

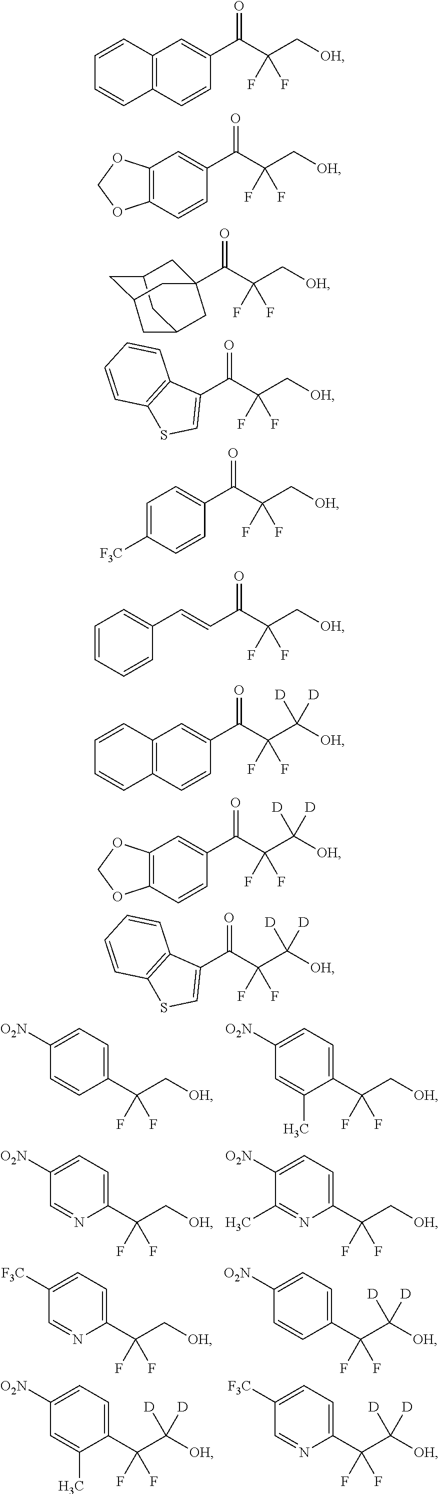

or any combination thereof.

Aspect 20. A solvent or co-solvent for organic synthesis comprising the substituted 2,2-difluoroethanol of any one of aspects 11-19.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: General Procedures

Experiments requiring anhydrous conditions were performed under argon atmosphere and organic solvents were dried over molecular sieves. All solvents and reagents were purchased from commercial sources unless otherwise noted. Gem-diols used as starting materials for compounds 1-5 and 13-18 were prepared according to previously published methods. Thin-layer chromatography was conducted using MilliporeSigma TLC silica gel 60 F254 plates. Preparative thin-layer chromatography was performed using Sorbent Technologies silica G prep TLC plates with UV254. Flash chromatography was conducted using SiliCycle Siliaflash silica gel P60 (40-63 μm) 60 Å. Melting points were taken on an OptiMelt apparatus from Stanford Research Systems and are not corrected. NMR spectra were recorded on a Bruker ARX 300 MHz, a Bruker Topspin Avance III HD 500 MHz spectrometer equipped with prodigy cryoprobe, or a Bruker Avance III HD 400 MHz spectrometer. The residual solvent peaks were used as an internal standard for ¹H and ¹³C NMR spectra, while trifluorotoluene was used as an added internal standard for ¹⁹F NMR spectra. Mass spectra were acquired by the Department of Chemistry at the University of Mississippi using SYNAPT HD Mass Spectrometer from Waters. Infrared spectra were recorded on Agilent Technologies Cary 630 FTIR.

### Example 2: Synthesis and Characterization of Compounds

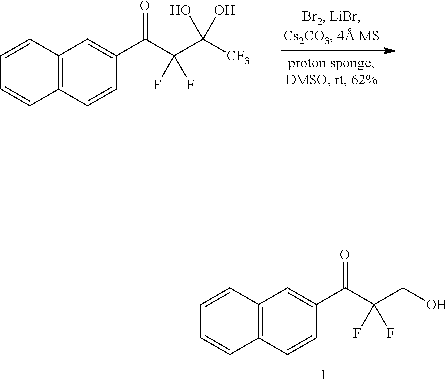 [SMILES: O=C(c1ccc2ccccc2c1)C(F)(F)C(O)(O)C(F)(F)F ; O=C(c1ccc2ccccc2c1)C(F)(F)CO]

2,2-Difluoro-3-hydroxy-1-(naphthalen-2-yl)propan-1-one (1). A mixture of activated 4 Å molecular sieves (100 mg), Cs₂CO₃ (106 mg, 0.325 mmol), LiBr (14 mg, 0.16 mmol), and proton sponge (14 mg, 0.07 mmol) in DMSO (1.0 mL) was cooled to 10° C. and treated with the dropwise addition of bromine (15 μL, 0.29 mmol). After 15 min, a solution of the gem-diol (10 mg, 0.031 mmol) in DMSO (0.3 mL) was added to the reaction mixture across 1 h via syringe pump. The reaction mixture was then stirred for 12 h at rt. Next, the reaction mixture was treated with saturated aqueous NH₄Cl (2 mL), diluted EtOAc (50 mL), and stirred for 1 h. The resultant mixture was filtered through Celite, and the organics were washed with brine (3×5 mL), dried over Na₂SO₄, and concentrated under reduced pressure. Preparative TLC (3% EtOAc in CHCl₃) afforded the title compound 1 as a pale yellow solid (4.5 mg, 62%): mp 42-44° C.; ¹H NMR (500 MHz, CDCl₃) δ 8.75 (s, 1H), 8.10 (d, J=8.5 Hz, 1H), 8.02 (d, J=8.1 Hz, 1H), 7.93 (d, J=8.7 Hz, 1H), 7.90 (d, J=8.1 Hz, 1H), 7.66 (ddd, J=8.3, 7.0, 1.5 Hz, 1H), 7.59 (ddd, J=8.0, 7.0, 1.0 Hz, 1H), 4.22 (td, J=12.9, 7.5 Hz, 2H), 2.39 (br s, 1H); ¹³C NMR (125 MHz, CDCl₃) δ 189.6 (t, J_{CF}=30.8 Hz, 1C), 136.2, 133.3 (t, J_{CF}=4.5 Hz, 1C), 132.2, 130.2, 129.6, 128.8 (t, J_{CF}=3.1 Hz, 1C), 128.7, 127.8, 127.1, 124.5, 116.2 (t, J_{CF}=257.0 Hz, 1C), 62.8 (t, J_{CF}=28.8 Hz, 1C); ¹⁹F NMR (376 MHz, CDCl₃) δ −109.1 (t, J_{HF}=12.8 Hz, 2F); IR (film) vₘₐₓ 3390, 3062, 2924, 2853, 1692, 1626, 1597, 1467 cm⁻¹; HRMS (ESI-TOF) m/z calcd for C₁₃H₉F₂O₂ [M−H]⁻ 235.0571, found 235.0563.

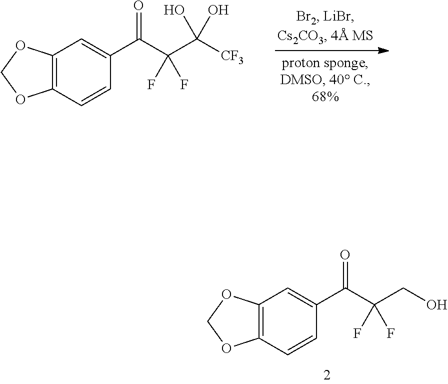 [SMILES: O=C(c1ccc2c(c1)OCO2)C(F)(F)C(O)(O)C(F)(F)F ; O=C(c1ccc2c(c1)OCO2)C(F)(F)CO]

1-(Benzo[1,3]dioxol-5-yl)-2,2-difluoro-3-hydroxypropan-1-one (2). A mixture of activated 4 Å molecular sieves (150 mg), Cs₂O₃ (135 mg, 0.414 mmol), LiBr (17 mg, 0.20 mmol), and proton sponge (17 mg, 0.079 mmol) in DMSO (1.5 mL) was cooled to 10° C. and treated with the dropwise addition of bromine (20 μL, 0.39 mmol). After 15 min, a solution of the gem-diol (12 mg, 0.038 mmol) in DMSO (1.0 mL) was added to the reaction mixture across 1 h via syringe pump. The reaction mixture was then stirred for 20 h at 40° C. Next, the reaction mixture was cooled to rt, treated with saturated aqueous NH₄Cl (3 mL), diluted EtOAc (50 mL), and stirred for 1 h. The resultant mixture was filtered through Celite, and the organics were washed with brine (3×5 mL), dried over Na₂SO₄, and concentrated under reduced pressure. Preparative TLC (10% EtOAc in CHCl₃) afforded the title compound 2 as a yellow oil (6 mg, 68%): ¹H NMR (500 MHz, CDCl₃) δ 7.81 (dd, J=8.4, 1.5 Hz, 1H), 7.54 (s, 1H), 6.90 (d, J=8.3 Hz, 1H), 6.08 (s, 2H), 4.12 (t, J=12.9Hz, 2H), 2.49 (s, 1H); ¹³C NMR (125 MHz, CDCl₃) δ 187.7 (t, J_{CF}=30.4 Hz, 1C), 153.4, 148.2, 127.7 (t, J_{CF}=4.3 Hz, 1C), 126.1 (t, J_{CF}=3.1 Hz, 1C), 116.2 (t, J_{CF}=257.3 Hz, 1C), 109.6 (t, J_{CF}=2.7 Hz, 1C), 108.3, 102.2, 62.7 (t, J_{CF}=29.1 Hz, 1C); ¹⁹F NMR (471 MHz, CDCl₃) δ −108.0 (t, J_{HF}=13.0 Hz, 2F); IR (film) vₘₐₓ 3463, 2916, 1685, 1606, 1491, 1448, 1359, 1255 cm⁻¹; HRMS (ESI-TOF) m/z calcd for C₁₀H₇F₂O₄ [M−H]⁻ 229.0312, found 229.0292.

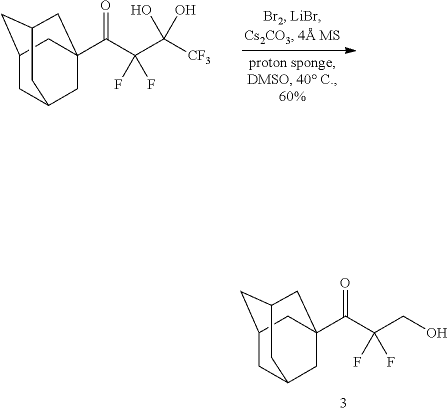 [SMILES: O=C(C12CC3CC(CC(C3)C1)C2)C(F)(F)C(O)(O)C(F)(F)F ; O=C(C(F)(F)CO)C12CC3CC(CC(C3)C1)C2]

1-(Adamantan-1-yl)-2,2-difluoro-3-hydroxypropan-1-one (3). A mixture of activated 4 Å molecular sieves (200 mg), Cs₂CO₃ (210 mg, 0.645 mmol), LiBr (25 mg, 0.29 mmol), and proton sponge (25 mg, 0.12 mmol) in DMSO (2 mL) was cooled to 10° C. and treated with the dropwise addition of bromine (30 μL, 0.58 mmol). After 15 min, a solution of the gem-diol (20 mg, 0.06 mmol) in DMSO (0.3 mL) was added to the reaction mixture across 30 min via syringe pump. The reaction mixture was then stirred for 12 h at 40° C. Next, the reaction mixture was cooled to rt, treated with saturated aqueous NH₄Cl (4 mL), diluted EtOAc (50 mL), and stirred for 1 h. The resultant mixture was filtered through Celite, and the organics were washed with brine (3×5 mL), dried over Na₂SO₄, and concentrated under reduced pressure. Preparative TLC (1% Et₂O in CH₂Cl₂) afforded the title compound 3 as a yellow oil (9 mg, 60%): ¹H NMR (500 MHz, CDCl₃) δ 3.94 (t, J=12.9 Hz, 2H), 2.08 (br s, 3H), 1.99 (br s, 6H), 1.75 (dd, J=20.5, 12.5 Hz, 6H), 1.61 (br s, 1H); ¹³C NMR (125 MHz, CDCl₃) δ 204.7 (t, J_{CF}=27.7 Hz, 1C), 116.9 (t, J_{CF}=258.7 Hz, 1C), 62.8 (t, J_{CF}=29.2 Hz, 1C), 46.3 (t, J_{CF}=2.6 Hz, 1C), 37.2 (3C), 36.3 (3C), 27.8 (3C); ¹⁹F NMR (471 MHz, CDCl₃) δ −111.3 (t, J_{HF}=13.0 Hz, 2F); IR (film) vₘₐₓ 3408, 2906, 2853, 1715, 1454 cm⁻¹; HRMS (ESI-TOF) m/z calcd for C₁₃H₁₇F₂O₂ [M−H]⁻ 243.1197, found 243.1193.

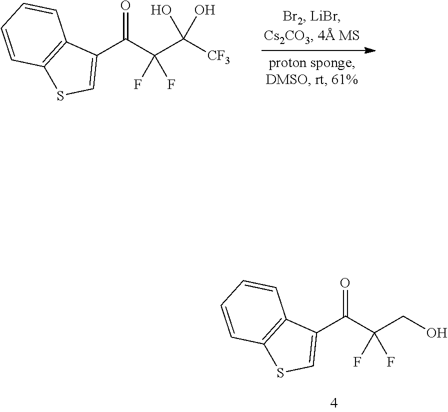 [SMILES: O=C(c1csc2ccccc12)C(F)(F)C(O)(O)C(F)(F)F ; O=C(c1csc2ccccc12)C(F)(F)CO]

1-(Benzothiophen-3-yl)-2,2-difluoro-3-hydroxypropan-1-one (4). A mixture of activated 4 Å molecular sieves (200 mg), Cs₂O₃ (220 mg, 0.675 mmol), LiBr (27 mg, 0.31 mmol), and proton sponge (27 mg, 0.13 mmol) in DMSO (2 mL) was cooled to 10° C. and treated with the dropwise addition of bromine (32 μL, 0.62 mmol). After 15 min, a solution of the gem-diol (20 mg, 0.061 mmol) in DMSO (1.0 mL) was added to the reaction mixture across 30 min via syringe pump. The reaction mixture was then stirred for 20 h at rt. Next, the reaction mixture was treated with saturated aqueous NH₄Cl (4 mL), diluted EtOAc (50 mL), and stirred for 1 h. The resultant mixture was filtered through Celite, and the organics were washed with brine (3×5 mL), dried over Na₂SO₄, and concentrated under reduced pressure. Preparative TLC (20% acetone in hexanes) afforded the title compound 4 as a brown oil (9 mg, 61%): ¹H NMR (500 MHz, CDCl₃) δ 8.80 (t, J=1.8 Hz, 1H), 8.72 (d, J=8.3 Hz, 1H), 7.90 (d, J=8.0 Hz, 1H), 7.54 (ddd, J=8.4, 7.2, 1.2 Hz, 1H), 7.47 (ddd, J=8.3, 7.1, 1.3 Hz, 1H), 4.20 (t, J=12.9 Hz, 2H), 2.47 (s, 1H); ¹³C NMR (125 MHz, CDCl₃) δ 184.3 (t, J_{CF}=30.1 Hz, 1C), 142.6 (t, J_{CF}=8.4 Hz, 1C), 139.0, 136.9, 128.6 (t, J_{CF}=2.8 Hz, 1C), 126.4, 126.0, 125.1, 122.3, 116.1 (t, J_{CF}=257.0 Hz, 1C), 62.6 (t, J_{CF}=29.0 Hz, 1C); ¹⁹F NMR (471 MHz, CDCl₃) δ −107.9 (t, J_{HF}=12.9 Hz, 2F); IR (film) vₘₐₓ 3423, 3119, 2929, 1677, 1490, 1460, 1174 cm⁻¹; HRMS (ESI-TOF) m/z calcd for C₁₁H₇F₂O₂S [M−H]⁻ 241.0135, found 241.0110.

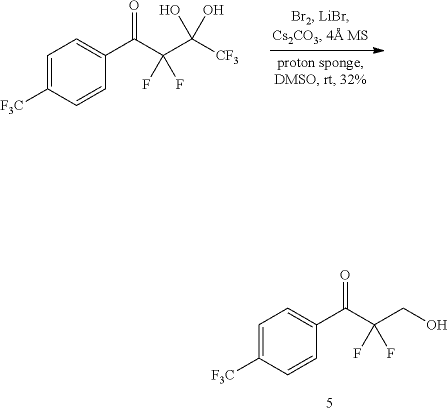 [SMILES: O=C(c1ccc(C(F)(F)F)cc1)C(F)(F)C(O)(O)C(F)(F)F ; O=C(c1ccc(C(F)(F)F)cc1)C(F)(F)CO]

2,2-Difluoro-3-hydroxy-1-(4-(trifluoromethyl)phenyl)propan-1-one (5). A mixture of activated 4 Å molecular sieves (300 mg), Cs₂O₃ (290 mg, 0.89 mmol), LiBr (38 mg, 0.44 mmol), and proton sponge (38 mg, 0.18 mmol) in DMSO (3 mL) was cooled to 10° C. and treated with the dropwise addition of bromine (38 μL, 0.74 mmol). After 15 min, a solution of the gem-diol (25 mg, 0.074 mmol) in DMSO (1.0 mL) was added to the reaction mixture across 1 h via syringe pump. The reaction mixture was then stirred for 12 h at rt. Next, the reaction mixture was treated with saturated aqueous NH₄Cl (6 mL), diluted EtOAc (50 mL), and stirred for 1 h. The resultant mixture was filtered through Celite, and the organics were washed with brine (3×5 mL), dried over Na₂SO₄, and concentrated under reduced pressure. Preparative TLC (6% EtOAc in CHCl₃) afforded the title compound 5 as a yellow oil (6 mg, 32%): ¹H NMR (500 MHz, CDCl₃) δ 8.24 (d, J=8.2 Hz, 2H), 7.79 (d, J=8.3 Hz, 2H), 4.18 (t, J=12.9 Hz, 2H), 2.34 (br s, 1H); ¹³C NMR (125 MHz, CDCl₃) δ 188.9 (t, J_{CF}=31.5 Hz, 1C), 135.7 (q, J_{CF}=32.9 Hz, 1C), 134.4, 130.5 (t, J_{CF}=3.2 Hz, 2C), 125.8 (q, J_{CF}=3.8 Hz, 2C), 123.2 (q, J_{CF}=273.8 Hz, 1C), 115.9 (t, J_{CF}=256.4 Hz, 1C), 62.3 (t, J_{CF}=28.8 Hz, 1C); ¹⁹F NMR (471 MHz, CDCl₃) δ −63.5 (s, 3F), −109.3 (t, J_{HF}=12.8 Hz, 2F); IR (film) vₘₐₓ 3362, 2867, 1709, 1413, 1329, 1174 cm⁻¹; HRMS (ESI-TOF) m/z calcd for C₁₀H₆F₅O₂ [M−H]⁻ 253.0288, found 253.0287.

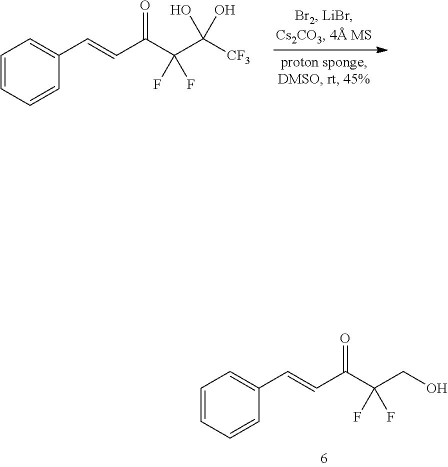 [SMILES: O=C(/C=C/c1ccccc1)C(F)(F)C(O)(O)C(F)(F)F ; O=C(/C=C/c1ccccc1)C(F)(F)CO]

(E)-4,4-Difluoro-5-hydroxy-1-phenylpent-1-en-3-one (6), A mixture of activated 4 Å molecular sieves (150 mg), Cs₂CO₃ (153 mg, 0.47 mmol), LiBr (20 mg, 0.23 mmol), and proton sponge (20 mg, 0.09 mmol) in DMSO (1.5 mL) was cooled to 10° C. and treated with the dropwise addition of bromine (15 μL, 0.29 mmol). After 15 min, a solution of the gem-diol (14 mg, 0.047 mmol) in DMSO (0.5 mL) was added to the reaction mixture across 1 h via syringe pump. The reaction mixture was then stirred for 12 h at rt. Next, the reaction mixture was treated with saturated aqueous NH₄Cl (3 mL), diluted EtOAc (25 mL), and stirred for 1 h. The resultant mixture was filtered through Celite, and the organics were washed with brine (3×5 mL), dried over Na₂SO₄, and concentrated under reduced pressure. Preparative TLC (6% EtOAc in CHCl₃) afforded the title compound 6 as a yellow oil (4.5 mg, 45%): ¹H NMR (500 MHz, CD₂Cl₂) δ 7.91 (d, J=16.0 Hz, 1H), 7.67 (d, J=6.4 Hz, 2H), 7.49 (m, 1H), 7.46 (d, J=7.2 Hz, 2H), 7.19 (d, J=16.0 Hz, 1H), 4.05 (t, J=12.8 Hz, 2H), 2.05 (s, 1H); ¹³C NMR (125 MHz, CD₂Cl₂) δ 189.1 (t, J_{CF}=30.0 Hz, 1C), 148.1, 134.0, 131.8, 129.2 (2C), 129.1 (2C), 118.1, 115.7 (t, J_{CF}=254.9 Hz, 1C), 62.2 (t, J_{CF}=29.0 Hz, 1C); ¹⁹F NMR (471 MHz, CDCl₃) δ −115.8 (t, J_{HF}=13.0 Hz, 2F); IR (film) vₘₐₓ 3388, 2958, 2926, 1701, 1610, 1451, 1329, 1181 cm⁻¹; HRMS (ESI-TOF) m/z calcd for C₁₁H₉F₂O₂ [M−H]⁻ 211.0571, found 211.0580.

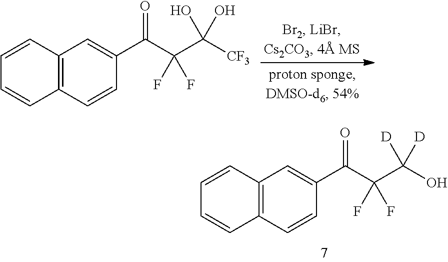 [SMILES: O=C(c1ccc2ccccc2c1)C(F)(F)C(O)(O)C(F)(F)F ; [2H]C([2H])(O)C(F)(F)C(=O)c1ccc2ccccc2c1]

3,3-Dideutero-2,2-difluoro-3-hydroxy-1-(naphthalen-2-yl)propan-1-one (7). A mixture of activated 4 Å molecular sieves (200 mg), LiBr (27 mg, 0.31 mmol), proton sponge (27 mg, 0.13 mmol), Cs₂CO₃ (242 mg, 0.743 mmol) in DMSO-d₆ (1.6 mL) was cooled to 10° C. and treated with the dropwise addition of bromine (32 uL, 0.62 mmol). After 15 min, a solution of the gem-diol (20 mg, 0.062 mmol) in DMSO-d₆ (0.5 mL) was added to the reaction mixture across 1 h via syringe pump. The reaction mixture was then stirred for 12 h at rt. Next, the reaction mixture was cooled to rt, treated with saturated aqueous NH₄Cl (5 mL), diluted EtOAc (50 mL), and stirred for 1 h. The resultant mixture was filtered through Celite, and the organics were washed with brine (3×5 mL), dried over Na₂SO₄, and concentrated under reduced pressure. Preparative TLC (5% EtOAc in CHCl₃) afforded the title compound 7 as a colorless solid (8 mg, 54%): mp 43-44° C.; ¹H NMR (500 MHz, CDCl₃) δ 8.75 (s, 1H), 8.10 (d, J=8.5 Hz, 1H), 8.01 (d, J=8.1 Hz, 1H), 7.93 (d, J=8.7 Hz, 1H), 7.89 (d, J=8.1 Hz, 1H), 7.66 (t, J=7.8 Hz, 1H), 7.59 (t, J=7.5 Hz, 1H), 2.34 (s, 1H); ¹³C NMR (125 MHz, CDCl₃) δ 189.6 (t, J_{CF}=30.5 Hz, 1C), 136.2, 133.3 (t, J_{CF}=4.5 Hz, 1C), 132.2, 130.2, 129.6, 128.9 (t, J_{CF}=3.0 Hz, 1C), 128.7, 127.8, 127.2, 124.5, 116.2 (t, J_{CF}=257.0 Hz, 1C), 62.8 (m, 1C); ¹⁹F NMR (471 MHz, CDCl₃) δ −108.4 (s, 2F); IR (film) vₘₐₓ 3439, 3062, 2958, 2928, 2864, 1689, 1627, 1467, 1296, 1371 cm⁻¹; HRMS (ESI-TOF) m/z calcd for C₁₃H₇D₂F₂O₂ [M−H]⁻ 237.0696, found 237.0710.

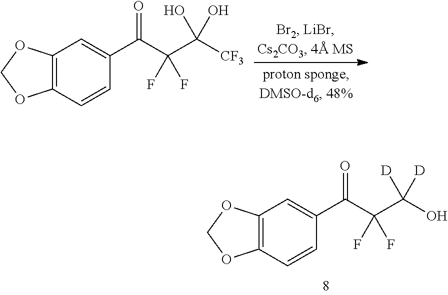 [SMILES: O=C(c1ccc2c(c1)OCO2)C(F)(F)C(O)(O)C(F)(F)F ; [2H]C([2H])(O)C(F)(F)C(=O)c1ccc2c(c1)OCO2]

3,3-Dideutero-1-(benzo[1,3]dioxol-5-yl)-2,2-difluoro-3-hydroxypropan-1-one (8). A mixture of activated 4 Å molecular sieves (150 mg), LiBr (19 mg, 0.22 mmol), proton sponge (19 mg, 0.18 mmol), Cs₂CO₃ (160 mg, 0.492 mmol) in DMSO-d₆ (1.7 mL) was cooled to 10° C. and treated with the dropwise addition of bromine (32 μL, 0.62 mmol). After 15 min, a solution of the gem-diol (14 mg, 0.044 mmol) in DMSO-d₆ (1.0 mL) was added to the reaction mixture across 1 h via syringe pump. The reaction mixture was then stirred for 24 h at 40° C. Next, the reaction mixture was cooled to rt, treated with saturated aqueous NH₄Cl (5 mL), diluted EtOAc (50 mL), and stirred for 1 h. The resultant mixture was filtered through Celite, and the organics were washed with brine (3 ×5 mL), dried over Na₂SO₄, and concentrated under reduced pressure. Preparative TLC (30% acetone in hexanes) afforded the title compound 8 as a yellow oil (5 mg, 48%): ¹H NMR (500 MHz, CDCl₃) δ 7.82 (d, J=10.1 Hz, 1H), 7.55 (s, 1H), 6.90 (d, J=8.4 Hz, 1H), 6.08 (s, 2H), 2.44 (s, 1H); ¹³C NMR (125 MHz, CDCl₃) δ 187.7 (t, J_{CF}=30.4 Hz, 1C), 153.4, 148.2, 127.7 (t, J_{CF}=4.3 Hz, 1C), 126.1 (t, J_{CF}=3.2 Hz, 1C), 116.0 (t, J_{CF}=257.0 Hz, 1C), 109.5 (t, J_{CF}=2.7 Hz, 1C), 108.3, 102.2, 62.7 (m, 1C); ¹⁹F NMR (471 MHz, CDCl₃) δ −108.2 (s, 2F); IR (film) vₘₐₓ 3420, 2916, 1681, 1605, 1446, 1247 cm⁻¹; HRMS (ESI-TOF) m/z calcd for C₁₀H₆D₂F₂O₄ (M+H)⁺ 233.0594, found 233.0595.

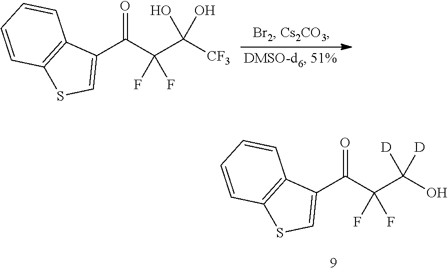 [SMILES: O=C(c1csc2ccccc12)C(F)(F)C(O)(O)C(F)(F)F ; [2H]C([2H])(O)C(F)(F)C(=O)c1csc2ccccc12]

3,3-Dideutero-1-(benzothiophen-3-yl)-2,2-difluoro-3-hydroxypropan-1-one (9). A mixture of activated 4 Å molecular sieves (250 mg), LiBr (35 mg, 0.40 mmol), proton sponge (35 mg, 0.16 mmol), Cs₂CO₃ (285 mg, 0.875 mmol) in DMSO-d₆ (3 mL) was cooled to 10° C. and treated with the dropwise addition of bromine (40 μL, 0.78 mmol). After 15 min, a solution of the gem-diol (26 mg, 0.080 mmol) in DMSO-d₆ (1.0 mL) was added to the reaction mixture across 1 h via syringe pump. The reaction mixture was then stirred for 24 h at 40° C. Next, the reaction mixture was cooled to rt, treated with saturated aqueous NH₄CI (5 mL), diluted EtOAc (50 mL), and stirred for 1 h. The resultant mixture was filtered through Celite, and the organics were washed with brine (3×5 mL), dried over Na₂SO₄, and concentrated under reduced pressure. Preparative TLC (5% EtOAc in hexanes) afforded the title compound 9 as a brown oil (10 mg, 51%): ¹H NMR (500 MHz, CDCI₃) δ 8.80 (t, J=1.8 Hz, 1H), 8.72 (d, J=8.9 Hz, 1H), 7.90 (d, J=8.0 Hz, 1H), 7.54 (t, J=7.7 Hz, 1H), 7.47 (t, J=7.6 Hz, 1H), 2.33 (s, 1H); ¹³C NMR (125 MHz, CDCI₃) δ 184.3 (t, J_{CF}=30.1 Hz, 1C), 142.7 (t, J_{CF}=8.4 Hz, 1C), 139.0, 136.9, 128.5 (t, J_{CF}=3.0 Hz, 1C), 126.4, 126.0, 125.1, 122.3, 116.0 (t, J_{CF}=257.0 Hz, 1C), 62.1 (m, 1C); ¹⁹F NMR (471 MHz, CDCI₃) δ −108.0 (s, 2F); IR (film) vₘₐₓ 3405, 3119, 3063, 1673, 1490, 1219, 1100 cm⁻¹; HRMS (ESI-TOF) m/z calcd for C₁₁H₅D₂F₂O₂S (M−H)⁻, 243.0260, found 243.0254.

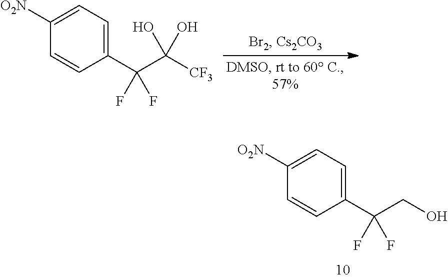 [SMILES: O=[N+]([O-])c1ccc(C(F)(F)C(O)(O)C(F)(F)F)cc1 ; O=[N+]([O-])c1ccc(C(F)(F)CO)cc1]

2,2-Difluoro-2-(4-nitrophenyl) ethan-1-ol (10). A mixture of activated 4 Å molecular sieves (130 mg) and Cs₂CO₃ (115 mg, 0.350 mmol) in DMSO (1.2 mL) was cooled to 10° C. and treated with the dropwise addition of bromine (17 μL, 0.33 mmol). After 15 min, a solution of the gem-diol (10 mg, 0.035 mmol) in DMSO (0.3 mL) was added to the reaction mixture across 1 h via syringe pump. The reaction mixture was then stirred for 18 h at 60° C. Next, the reaction mixture was cooled to rt, treated with saturated aqueous NH₄Cl (2 mL), diluted EtOAc (50 mL), and stirred for 1 h. The resultant mixture was filtered through Celite, and the organics were washed with brine (3×5 mL), dried over Na₂SO₄, and concentrated under reduced pressure. Preparative TLC (3% EtOAc in CHCl₃) afforded the title compound 10 as a colorless solid (4 mg, 57%): mp 86-89° C.; ¹H NMR (500 MHz, CDCl₃) δ 8.31 (d, J=8.4 Hz, 2H), 7.74 (d, J=8.6 Hz, 2H), 4.02 (t, J=12.7 Hz, 2H), 1.84 (br s, 1H); ¹³C NMR (125 MHz, CDCl₃) δ 149.4, 140.7 (t, J_{CF}=26.1 Hz, 1C), 127.0 (t, J_{CF}=6.1 Hz, 2C), 123.7 (2C), 119.6 (t, J_{CF}=253.1 Hz, 1C), 65.6 (t, J_{CF}=32.9 Hz, 1C); ¹⁹F NMR (376 MHz, CDCl₃) δ −107.1 (t, J_{HF}=12.8 Hz, 2F); IR (film) vₘₐₓ 3519, 1519, 1352, 1314 cm⁻¹; HRMS (EI-BE) m/z calcd for C₈H₇F₂NO₃ [M]⁺ 203.0394, found, 203.0392.

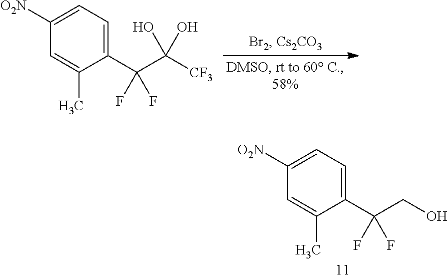 [SMILES: Cc1cc([N+](=O)[O-])ccc1C(F)(F)C(O)(O)C(F)(F)F ; Cc1cc([N+](=O)[O-])ccc1C(F)(F)CO]

2,2-Difluoro-2-(2-methyl-4-nitrophenyl)ethan-1-ol (11). A mixture of activated 4 Å molecular sieves (170 mg) and Cs₂CO₃ (130 mg, 0.40 mmol) in DMSO (1.5 mL) was cooled to 10° C. and treated with the dropwise addition of bromine (20 μL, 0.39 mmol). After 15 min, a solution of the gem-diol (12 mg, 0.04 mmol) in DMSO (0.5 mL) was added to the reaction mixture across 1 h via syringe pump. The reaction mixture was then stirred for 24 h at 65° C. Next, the reaction mixture was cooled to rt, treated with saturated aqueous NH₄Cl (3 mL), diluted EtOAc (50 mL), and stirred for 1 h. The resultant mixture was filtered through Celite, and the organics were washed with brine (3×5 mL), dried over Na₂SO₄, and concentrated under reduced pressure. Preparative TLC (3% EtOAc in CHCl₃) afforded the title compound 11 as a colorless solid (5 mg, 58%): mp 106-107° C.; ¹H NMR (500 MHz, CDCl₃) δ 8.09 (s, 1H), 8.08 (d, J=8.5 Hz, 1H), 7.69 (d, J=8.2 Hz, 1H), 4.06 (t, J=13.3 Hz, 2H), 2.59 (s, 3H), 1.96 (br s, 1H); ¹³C NMR (125 MHz, CDCl₃) δ 148.7, 138.8 (t, J_{CF}=2.3 Hz, 1C) 138.5 (t, J_{CF}=24.4 Hz, 1C), 128.4 (t, J_{CF}=8.7 Hz, 1C), 126.6, 120.8 (t, J_{CF}=246.1 Hz, 1C), 120.8, 64.9 (t, J_{CF}=31.7 Hz, 1C), 20.5 (t, J_{CF}=4.0 Hz, 1C); ¹⁹F NMR (471 MHz, CDCl₃) δ 105.0 (t, J_{HF}=13.4 Hz, 2F); IR (film) vₘₐₓ 3521, 3096, 2946, 1520, 1351 cm⁻¹; HRMS (ESI-TOF) m/z calcd for C₉H₈F₂NO₃ [M−H] 216.0472, found 216.0473.

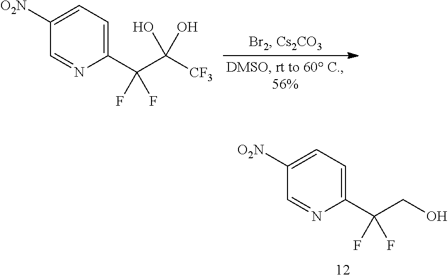 [SMILES: O=[N+]([O-])c1ccc(C(F)(F)C(O)(O)C(F)(F)F)nc1 ; O=[N+]([O-])c1ccc(C(F)(F)CO)nc1]

2,2-Difluoro-2-(5-nitropyridin-2-yl)ethan-1-ol (12). A mixture of activated 4 Å molecular sieves (100 mg) and Cs₂CO₃ (110 mg, 0.338 mmol) in DMSO (1.2 mL) was cooled to 10° C. and treated with the dropwise addition of bromine (15 μL, 0.29 mmol). After 15 min, a solution of the gem-diol (10 mg, 0.033 mmol) in DMSO (0.5 mL) was added to the reaction mixture across 1 h via syringe pump. The reaction mixture was then stirred for 24 h at 60° C. Next, the reaction mixture was cooled to rt, treated with saturated aqueous NH₄Cl (2 mL), diluted EtOAc (50 mL), and stirred for 1 h. The resultant mixture was filtered through Celite, and the organics were washed with brine (3×5 mL), dried over Na₂SO₄, and concentrated under reduced pressure. Preparative TLC (10% EtOAc in CHCl₃) afforded the title compound 12 as a colorless oil (4 mg, 56%): ¹H NMR (500 MHz, CDCl₃) δ 9.45 (s, 1H), 8.65 (dd, J=8.6, 2.6 Hz, 1H), 7.95 (d, J=8.6 Hz, 1H), 4.28 (t, J=12.5 Hz, 2H), 2.70 (br s, 1H); ¹³C NMR (125 MHz, CDCl₃) δ 158.5 (t, J_{CF}=30.1 Hz, 1C), 144.8, 144.6, 132.7, 121.8 (t, J_{CF}=3.6 Hz, 1C), 117.7 (t, J_{CF}=244.6 Hz, 1C), 63.7 (t, J_{CF}=30.7 Hz, 1C). ¹⁹F NMR (471 MHz, CDCl₃) δ −107.1 (t, J_{HF}=12.5 Hz, 2F); IR (film) vₘₐₓ 3585, 3109, 2936, 1607, 1530, 1358, 1320 cm⁻¹; HRMS (ESI-TOF) m/z calcd for C₇H₇F₂N₂O₃ [M+H]⁺ 205.0425, found 205.0424.

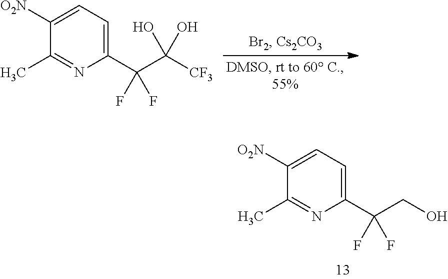 [SMILES: Cc1nc(C(F)(F)C(O)(O)C(F)(F)F)ccc1[N+](=O)[O-] ; Cc1nc(C(F)(F)CO)ccc1[N+](=O)[O-]]

2,2-Difluoro-2-(6-methyl-5-nitropyridin-2-yl)ethan-1-ol (13). A mixture of activated 4 Å molecular sieves (100 mg) and Cs₂CO₃ (110 mg, 0.338 mmol) in DMSO (1.0 mL) was cooled to 10° C. and treated with the dropwise addition of bromine (15 μL, 0.29 mmol). After 15 min, a solution of the gem-diol (10 mg, 0.033 mmol) in DMSO (0.4 mL) was added to the reaction mixture across 1 h via syringe pump. The reaction mixture was then stirred for 24 h at 60° C. Next, the reaction mixture was cooled to rt, treated with saturated aqueous NH₄Cl (2 mL), diluted EtOAc (50 mL), and stirred for 1 h. The resultant mixture was filtered through Celite, and the organics were washed with brine (3×5 mL), dried over Na₂SO₄, and concentrated under reduced pressure. Preparative TLC (10% EtOAc in CHCl₃) afforded the title compound 13 as a colorless solid (4 mg, 55%): mp 73-75° C.; ¹H NMR (400 MHz, CDCl₃) δ 8.42 (d, J=8.4 Hz, 1H), 7.78 (d, J=8.6 Hz, 1H), 4.27 (td, J=12.3, 6.2 Hz, 3H), 2.90 (s, 3H), 2.74 (t, J=6.8 Hz, 1H); ¹³C NMR (100 MHz, CDCl₃) δ 156.3 (t, J_{CF}=30.1 Hz, 1C), 153.6, 146.3, 134.0, 119.6 (t, J_{CF}=3.6 Hz, 1C) 117.5 (t, J_{CF}=244.9 Hz, 1C), 63.8 (t, J_{CF}=30.6 Hz, 1C), 23.8; ¹⁹F NMR (376 MHz, CDCl₃) δ −108.2 (t, J_{HF}=12.6 Hz, 2F); IR (film) vₘₐₓ 3400, 3094, 2934, 1602, 1582, 1530, 1350, 1332 cm⁻¹; HRMS (ESI-TOF) m/z calcd for C₈H₇F₂N₂O₃ [M−H]⁻ 217.0425, found 217.0415.

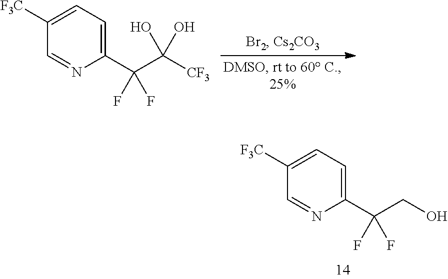 [SMILES: OC(O)(C(F)(F)F)C(F)(F)c1ccc(C(F)(F)F)cn1 ; OCC(F)(F)c1ccc(C(F)(F)F)cn1]

2,2-Difluoro-2-(5-(trifluoromethyl)pyridin-2-yl)ethan-1-ol (14). A mixture of activated 4 Å molecular sieves (200 mg) and Cs₂CO₃ (208 mg, 0.638 mmol) in DMSO (2.0 mL) was cooled to 10° C. and treated with the dropwise addition of bromine (30 μL, 0.59 mmol). After 15 min, a solution of the gem-diol (18 mg, 0.058 mmol) in DMSO (0.5 mL) was added to the reaction mixture across 1 h via syringe pump. The reaction mixture was then stirred for 24 h at 60° C. Next, the reaction mixture was cooled to rt, treated with saturated aqueous NH₄Cl (4 mL), diluted EtOAc (50 mL), and stirred for 1 h. The resultant mixture was filtered through Celite, and the organics were washed with brine (3×10 mL), dried over Na₂SO₄, and concentrated under reduced pressure. Preparative TLC (25% acetone in hexanes) afforded the title compound 14 as a colorless oil (4 mg, 25%): ¹H NMR (500 MHz, CDCl₃) δ 8.91 (s, 1H), 8.13 (dd, J=8.2 Hz, 1H), 7.88 (d, J=8.2 Hz, 1H), 4.27 (t, J=12.5 Hz, 2H), 2.83 (s, 1H); ¹³C NMR (125 MHz, CDCl₃) δ 156.9 (t, J_{CF}=29.6 Hz, 1C), 146.2 (q, J_{CF}=3.9 Hz, 1C), 135.1 (q, J_{CF}=3.7 Hz, 1C), 128.2 (q, J_{CF}=33.7 Hz, 1C), 122.9 (q, J_{CF}=272.5 Hz, 1C), 121.1 (t, J_{CF}=3.3 Hz, 1C), 117.6 (t, J_{CF}=244.0 Hz, 1C), 63.9 (t, J_{CF}=30.8 Hz, 1C); ¹⁹F NMR (471 MHz, CDCl₃) δ −62.7 (s, 3F), −106.9 (t, J_{HF}=12.6 Hz, 2F); IR (film) vₘₐₓ 3339, 2930, 2854, 1735, 1689, 1556, 1350, 1463, 1325 cm⁻¹; HRMS (ESI-TOF) m/z calcd for C₈H₇F₅NO [M+H]⁺ 228.0448, found 228.0457.

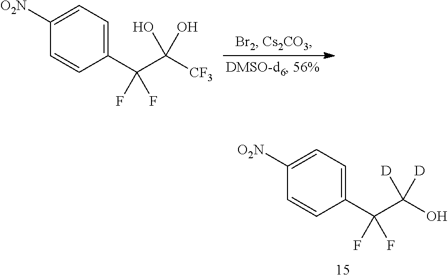 [SMILES: O=[N+]([O-])c1ccc(C(F)(F)C(O)(O)C(F)(F)F)cc1 ; [2H]C([2H])(O)C(F)(F)c1ccc([N+](=O)[O-])cc1]

1,1,-Dideutero-2,2-difluoro-2-(4-nitrophenyl)ethan-1-ol (15). A mixture of activated 4 Å molecular sieves (100 mg) and Cs₂O₃ (115 mg, 0.350 mmol) in DMSO-d₆ (1.2 mL) was cooled to 10° C. and treated with the dropwise addition of bromine (17 μL, 0.33 mmol). After 15 min, a solution of the gem-diol (10 mg, 0.035 mmol) in DMSO-d₆ (0.3 mL) was added to the reaction mixture across 1 h via syringe pump. The reaction mixture was then stirred for 24 h at 65° C. Next, the reaction mixture was cooled to rt, treated with saturated aqueous NH₄Cl (2 mL), diluted with EtOAc (50 mL), and stirred for 1 h. The resultant mixture was filtered through Celite, and the organics were washed with brine (3×5 mL), dried over Na₂SO₄, and concentrated under reduced pressure. Preparative TLC (5% EtOAc in CHCl₃) afforded the title compound 15 as a colorless solid (4 mg, 56%): mp 86-89° C.; ¹H NMR (500 MHz, CDCl₃) δ 8.31 (d, J=8.9 Hz, 2H), 7.73 (d, J=8.9 Hz, 2H), 2.17 (br s, 1H); ¹³C NMR (125 MHz, CDCl₃) δ 149.1, 140.7 (t, J_{CF}=26.1 Hz, 1C), 127.0 (t, J_{CF}=6.1 Hz, 2C), 123.7 (2C), 119.6 (t, J_{CF}=244.8 Hz, 1C), 64.9 (m, 1C); ¹⁹F NMR (471 MHz, CDCl₃) δ −107.3; IR (film) vₘₐₓ 3409, 3088, 2868, 1611, 1529, 1352, 1284, 1113 cm⁻¹; HRMS (ESI-TOF) m/z calcd for C₈H₄D₂F₂NO₃ [M−H] 204.0441, found 204.0418.

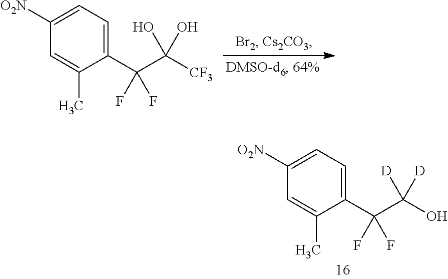 [SMILES: Cc1cc([N+](=O)[O-])ccc1C(F)(F)C(O)(O)C(F)(F)F ; [2H]C([2H])(O)C(F)(F)c1ccc([N+](=O)[O-])cc1C]

1,1-Dideutero-2,2-difluoro-2-(2-methyl-4-nitrophenyl)ethan-1-ol (16). A mixture of activated 4 Å molecular sieves (200 mg) and Cs₂O₃ (170 mg, 0.522 mmol) in DMSO-d₆ (2 mL) was cooled to 10° C. and treated with the dropwise addition of bromine (25 μL, 0.49 mmol). After 15 min, a solution of the gem-diol (15 mg, 0.05 mmol) in DMSO-d₆ (0.5 mL) was added to the reaction mixture across 1 h via syringe pump. The reaction mixture was then stirred for 24 h at 65° C. Next, the reaction mixture was cooled to rt, treated with saturated aqueous NH₄Cl (5 mL), diluted EtOAc (50 mL), and stirred for 1 h. The resultant mixture was filtered through Celite, and the organics were washed with brine (3×5 mL), dried over Na₂SO₄, and concentrated under reduced pressure. Preparative TLC (5% EtOAc in CHCl₃) afforded the title compound 16 as a colorless solid (7 mg, 64%): mp 107-108° C.; ¹H NMR (500 MHz, CDCl₃) δ 8.09 (s, 1H), 8.08 (d, J=8.3 Hz, 1H), 7.68 (d, J=8.2 Hz, 1H), 2.59 (s, 3H), 2.21 (br s, 1H); ¹³C NMR (125 MHz, CDCl₃) δ 148.7, 138.6 (t, J=2.3 Hz), 138.7 (t, J_{CF}=24.4 Hz, 1C), 128.3 (t, J_{CF}=8.6 Hz, 1C), 126.6, 121.0 (t, J_{CF}=245.9 Hz, 1C). 120.7, 64.3 (m, 1C), 20.4 (t, J_{CF}=4.1 Hz, 1C); ¹⁹F NMR (471 MHz, CDCl₃) δ −104.3 (s, 2F); IR (film) vₘₐₓ 3521, 3096, 2946, 1520, 1351 cm⁻¹; HRMS (ESI-TOF) m/z calcd for C₉H₆F₂D₂NO₃ [M−H]⁻ 218.0598, found 218.0605.

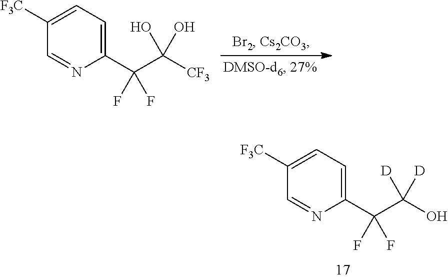 [SMILES: OC(O)(C(F)(F)F)C(F)(F)c1ccc(C(F)(F)F)cn1 ; [2H]C([2H])(O)C(F)(F)c1ccc(C(F)(F)F)cn1]

1,1-Dideutero-2,2-difluoro-2-(5-(trifluoromethyl)pyridin-2-yl)ethan-1-ol (17). A mixture of activated 4 Å molecular sieves (150 mg) and Cs₂CO₃ (208 mg, 0.638 mmol) in DMSO-d₆ (2 mL) was cooled to 10° C. and treated with the dropwise addition of bromine (25 μL, 0.48 mmol). After 15 min, a solution of the gem-diol (15 mg, 0.05 mmol) in DMSO-d₆ (0.5 mL) was added to the reaction mixture across 1 h via syringe pump. The reaction mixture was then stirred for 24 h at 65° C. Next, the reaction mixture was cooled to rt, treated with saturated aqueous NH₄Cl (5 mL), diluted EtOAc (50 mL), and stirred for 1 h. The resultant mixture was filtered through Celite, and the organics were washed with brine (3×5 mL), dried over Na₂SO₄, and concentrated under reduced pressure. Preparative TLC (4% Et₂O in CHCl₃) afforded the title compound 17 as a colorless oil (3 mg, 27%): ¹H NMR (500 MHz, CDCl₃) δ 8.91 (d, J=2.5 Hz, 1H), 8.12 (d, J=8.2 Hz, 1H), 7.88 (d, J=8.2 Hz, 1H), 2.76 (br s, 1H); ¹³C NMR (125 MHz, CDCl₃) δ 156.9 (t, J_{CF}=30.2 Hz, 1C), 146.1 (q, J=4.1 Hz, 1C), 135.0 (q, J=3.6 Hz, 1C), 128.3 (q, J_{CF}=33.5 Hz, 1C), 122.9 (q, J_{CF}=272.9 Hz, 1C), 121.1 (t, J_{CF}=3.4 Hz), 117.8 (t, J_{CF}=243.9 Hz, 1C), 63.3 (m, 1C); ¹⁹F NMR (471 MHz, CDCl₃) δ −62.8 (s, 3F), −107.1 (s, 2F); IR (film) vₘₐₓ 3343, 2918, 2850, 1724, 1609, 1398, 1332, 1143 cm⁻¹; HRMS (ESI-TOF) m/z calcd for C₈H₅D₂F₅NO [M+H]⁺ 230.0573, found 230.0572.

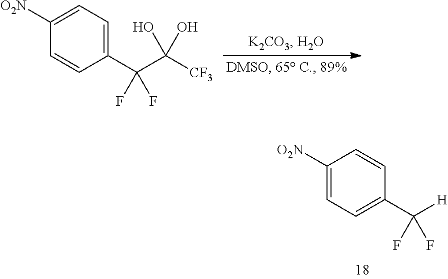 [SMILES: O=[N+]([O-])c1ccc(C(F)(F)C(O)(O)C(F)(F)F)cc1 ; O=[N+]([O-])c1ccc(C(F)F)cc1]

1-(Difluoromethyl)-4-nitrobenzene (18). A solution of the gem-diol (100 mg, 0.348 mmol) in DMSO (2.0 mL) was treated with H₂O (19 μL) and K₂CO₃ (194 mg, 1.40 mmol). The reaction mixture was stirred at 65° C. for 3 h. Next, the reaction mixture was cooled to rt, treated with saturated aqueous NH₄Cl (2 mL), and extracted with CH₂Cl₂ (5×5 mL). The organics were dried over Na₂SO₄ and concentrated under reduced pressure. SiO₂ flash column chromatography (8:2 hexanes/EtOAc) afforded the title compound 18 as a colorless oil (54 mg, 89%): ¹H NMR (300 MHz, CDCl₃) δ 8.33 (d, J=8.9 Hz, 2H), 7.72 (d, J=8.6 Hz, 2H), 6.65 (t, J=55.7 Hz, 1H); ¹⁹F NMR (282 MHz, CDCl₃) δ −114.0 (d, J_{HF}=55.7 Hz, 2F). All spectral and characterization data matched the reported data.

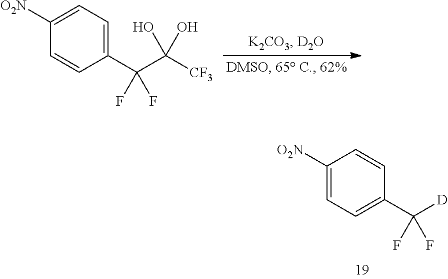 [SMILES: O=[N+]([O-])c1ccc(C(F)(F)C(O)(O)C(F)(F)F)cc1 ; [2H]C(F)(F)c1ccc([N+](=O)[O-])cc1]

1-(Deutrodifluoromethyl)-4-nitrobenzene (19), A solution of the gem-diol (100 mg, 0.348 mmol) in DMSO (2.0 mL) was treated with D₂O (19 μL) and K₂CO₃ (194 mg, 1.40 mmol). The reaction mixture was stirred at 65° C. for 30 min. Next, the reaction mixture was cooled to rt, treated with saturated aqueous NH₄Cl (2 mL), and extracted with CH₂Cl₂ (5×5 mL). The organics were dried over Na₂SO₄ and concentrated under reduced pressure. SiO₂ flash column chromatography (8:2 hexanes/EtOAc) afforded the title compound 19 as a colorless oil (38 mg, 62%): ¹H NMR (300 MHz, CDCl₃) δ 8.33 (d, J=9.0 Hz, 2H), 7.72 (d, J=9.0 Hz, 2H); ¹³C NMR (125 MHz, CDCl₃) δ 149.3, 140.1 (t, J_{CF}=22.7 Hz, 1C), 126.8 (t, J_{CF}=5.9 Hz, 2C), 124.0 (2C), 112.8 (tt, J_{CF}=238.8, 28.4 Hz, 1C); ¹⁹F NMR (282 MHz, CDCl₃) δ −114.7 (t, J_{DF}=8.5 Hz, 2F); IR (film) vₘₐₓ 1529, 1351, 1277 cm⁻¹; HRMS (CI-Q) m/z calcd. for C₇H₄DF₂NO₂ [M]⁺, 174.0351; found, 174.0353.

### Example 3: Computational Chemistry

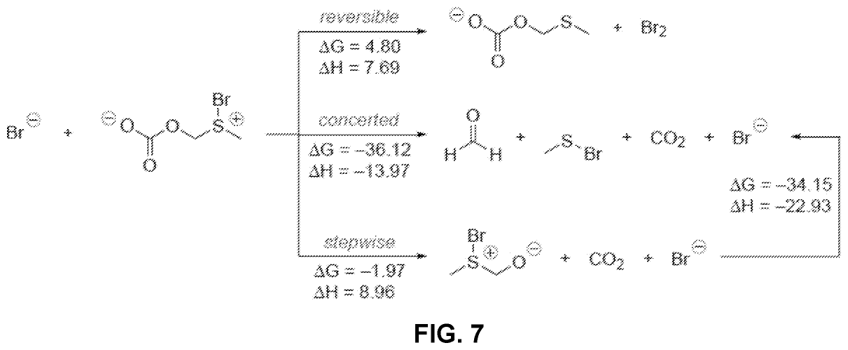

Geometry optimization and frequency calculations were performed with the Gaussian 097software package using hybrid density functional theory with the M06-2X level of theory and 6-311++G(3df, 3pd) basis set. Integral calculations utilized an UltraFine grid. and Cartesian coordinates were utilized in. all cases, except that a Z-matrix was used for (bromo(methyl)sulfonio)methanolate. Calculations included DMSO solvent effects using the Polarizable Continuum Model (PCM) with the integral equation formalism variant (IEFPCM). No imaginary frequencies were observed for the obtained minima. Free energies (G) and enthalpies (H) are reported in atomic units at 298.15 K (Table 1) and include the zero-point energy. The concerted fragmentation process has the lowest energies compared to both the reversible and the stepwise fragmentation processes (FIG. 7).

TABLE 1
Free Energies (G) and Enthalpies (H) of Intermediates and
Products in the Proposed Mechanism

| Compound | G (au) | H (au) |
|---|---|---|
| 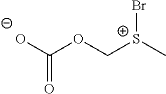 | −3315.295323 | −3315.247698 |
| 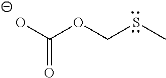 | −741.305751 | −741.262752 |
| Br₂ | −5148.358075 | −5148.330310 |
| 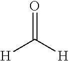 | −114.494958 | −114.470157 |
| CO₂ | −188.600670 | −188.576452 |
| 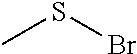 | −3012.257250 | −3012.223359 |
| 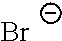 | −2574.376153 | −2574.357617 |
| 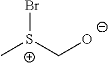 | −3126.697791 | −3126.656971 |

### Example 4: Results and Discussion

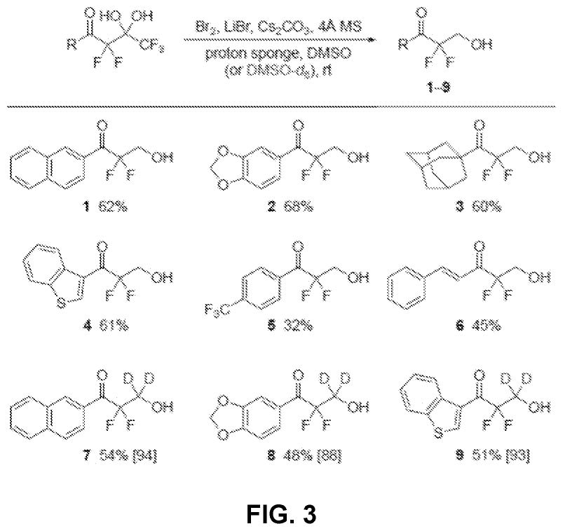

The production of difluorocarbanions from pentafluoro-gem-diols following the addition of K₂CO₃ in the solvent DMSO has previously been reported. The use of DMSO is an ideal starting point for the development of a method for hydroxymethylation of difluorocarbanions because this solvent is a known precursor of formaldehyde. Accordingly, it was determined that the addition of bromine and 4 Å molecular sieves along with K₂CO₃ in DMSO generates formaldehyde. These conditions were optimized for the simultaneous production of difluoroenolates from pentafluoro-gem-diols by adding LiBr and proton sponge. Lastly, K₂CO₃ was replaced with Cs₂CO₃ after screening other carbonate bases (e.g., Na₂CO₃ and Li₂CO₃). Using these conditions, the pentafluoro-gem-diols were hydroxymethylated and formed the products 1-6 in 32-68% isolated yields (FIG. 3). The higher conversions were observed with substrates bearing a naphthyl ring 1, a benzodioxoyl ring 2, an adamantyl group 3, or a benzothiophene 4, whereas the lower yields were obtained from the p-CF₃ benzene 5 and the styrene derivative 6. The incorporation of deuterium into the structure of organic molecules is a growing field, especially for metabolic probes, leads in drug discovery, and internal standards in analytical techniques. Moreover, the presence of both deuterium and fluorine atoms on organic structures is an under-explored area, and few synthetic strategies are available to create these types of molecules. In order to address this shortcoming, the disclosed method has been adapted for hydroxydeuteromethylation by exchanging DMSO with DMSO-d₆. The pentafluoro-gem-diols were subjected to these modified conditions (FIG. 3). The difluorodideuteroethanols 7-9 were synthesized with high levels of incorporation of deuterium (88-94%) in similar conversions as the hydroxymethylations.

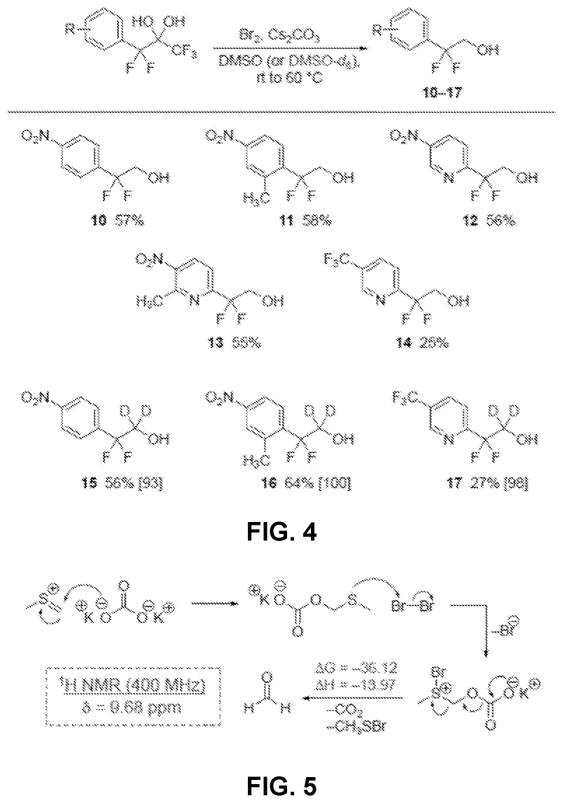

This process for hydroxymethylation could be simplified in the case of difluorobenzyl carbanions. Specifically, only Cs₂CO₃, bromine, and 4 Å molecular sieves were added in the presence of the pentafluoro-gem-diols shown in FIG. 4. The transformation produces the aryl substituted difluoroethanols displaying 4-nitrobenzenes 10-11 or 5-nitro-2-pyridines 12-13 in isolated yields of 55-58%. The 5-trifluoromethyl-2-pyridine adduct 14 was produced in a lower 25% yield, but this observation was anticipated from our previous findings. Also, the difluorodideuteroethanols 15-17 were synthesized, by replacing DMSO with DMSO-d₆, with high levels of incorporation of deuterium (93-100%) in similar yields as the respective hydroxymethylations.

A Pummerer-like process is expected in this hydroxymethylation reaction. A plausible mechanism is proposed that initiates with the generation of methyl(methylene)sulfonium cation (FIG. 5). Next, a carbonate base (e.g., K₂CO₃) serves as a nucleophile and adds to the electrophilic thionium cation. Then, the methylsulfide group is oxidized by bromine, and the resulting intermediate displaying the bromosulfonium ion fragments by the release of methanesulfenyl bromide and decarboxylation. The final result is the production of formaldehyde. Accordingly, after stirring K₂CO₃, Br₂, and 4 Å molecular sieves in DMSO at 60° C. for two hours, formaldehyde is observed in the ¹H NMR spectrum with the characteristic peak at 9.68 ppm (in acetone-d₆). Computational studies were performed using density functional theory in DMSO. The M06-2X level of theory and 6-311++G(3df,3pd) basis set were utilized. The free energies and enthalpy (i.e., ΔG=−36.12 and ΔH=−13.97 kcal/mol) are lower for the concerted fragmentation of the key bromosulfonium intermediate compared to both the reverse process, the regeneration of the (methylthio)methyl carbonate intermediate, and the stepwise fragmentation (FIG. 7). These results support a tandem fragmentation/decarboxylation process. The production of formaldehyde from this reaction allows the hydroxymethylation of the difluorobenzyl carbanion.

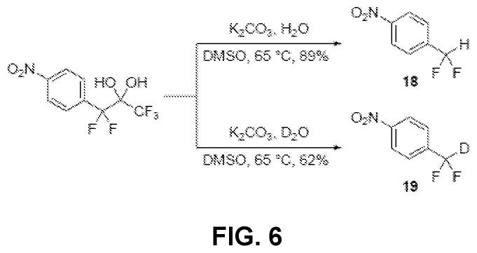

Additional mechanistic insight for this process was gathered from the reaction of 1,1,1,3,3-pentafluoro-3-(4-nitrophenyl)propane-2,2-diol with K₂CO₃ and H₂O in DMSO. First, if this reaction is conducted at 65° C. without the presence of an electrophile, such as formaldehyde, the 1-(difluoromethyl)-4-nitrobenzene 18 is produced (FIG. 6). Product 18 arises from the slow protonation of the difluorobenzyl carbanion. Second, if this same reaction is conducted with D₂O instead of H₂O, the 1-(deuterodifluoromethyl)-4-nitrobenzene 19 is isolated. Methods for the production of deuterodifluoromethylbenzenes are rare in the literature. Overall, the data obtained from the protonation and deuteration studies with the difluoromethylbenzenes compare favorably to our similar studies with difluoromethyl ketones.

In summary, disclosed herein is an approach for the hydroxymethylation and hydroxydeuteromethylation of difluoroenolates and difluorobenzyl carbanions generated from pentafluoro gem-diols. This process produces formaldehyde or formaldehyde-d₂ in the presence of a weak base, and in the latter case, high levels of deuterium incorporation are observed. The synthesis of a 2,2-difluoro-1,1-dideuteroethanol (i.e., RCF₂CD₂OH) has been previously reported via reduction of a difluorinated ester or amide using the deuterated reducing agents, NaBD₄ or LiAlHD₄; however, these reagents can concomitantly reduce other carbonyl or susceptible groups. No other preparations of 2,2-difluoro-1,1-dideuteroethanols exist in the literature; therefore, the disclosed methodology provides a viable option for these targets. A plausible mechanism for the hydroxymethylation is proposed and supported with experimental and computational studies; formaldehyde was observed by NMR. This strategy not only demonstrates new reactions for difluoroenolates and difluorobenzyl carbanions but also presents another method for the in situ formation of formaldehyde.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Alexanian, E. J. et al, Org. Lett. 2019, 21, 9268-9271.
2. Andersen, T. L. et al, Angew. Chem. Int. Ed., 2016, 55, 10396-10400.
3. Atzrodt, J. et al, Angew. Chem. Intl. Ed., 2018, 57, 1758-1784.
4. Balnaves, A. S. et al, J. Chem. Soc., Perkins Trans. 1, 1999, 2525-2535.
5. Barcan, G. A. et al, Org. Process. Res. Dev., 2019, 23, 1396-1406.
6. Becke, A. D. J. Chem. Phys., 2014, 140, 18A301.
7. Boeckman, R. K. Jr., et al, Org. Lett., 2018, 20, 5062-5065.
8. Bur, S. K. et al, Chem. Rev., 2004, 104, 2401-2432.
9. Forrester, J. et al, J. Chem. Soc. Perkin Trans. 1, 1995, 18, 2289-2291.
10. Frisch, M. J. et al, Gaussian 09, Revision D.01. Gaussian, Inc., Wallingford CT, 2013.
11. Gant, T. G. J. Med. Chem., 2014, 57, 3595-3611.
12. Gillis, E. P. et al, J. Med. Chem., 2015, 58, 8315-8359.
13. Han, C. Discovery of the Trifluoroacetate Release Process and Its Strategic Application Toward the Synthesis of Biologically Active Molecules. Ph.D. Dissertation, Purdue University: West Lafayette, IN, 2013.
14. Han, C. et al, J. Am. Chem. Soc., 2011, 130, 5802-5805.
15. John, J. P. et al, J. Org. Chem., 2011, 76, 9163-9168.
16. Khatri, H. R. et al, J. Org. Chem., 2019, 84, 11665-11675.
17. King, J. F. et al, J. Org. Chem., 1998, 63, 808-811.
18. Kutwal, M. S. et al, Org. Lett., 2019, 21, 2509-2513.
19. Marin-Valls, R. K. et al, ACS Catal., 2019, 9, 7568-7577.
20. Meng, Y. et al, J. Med. Chem., 2021, 64, 925-937.
21. Miao, W. Y. et al, J. Am. Chem. Soc., 2018, 140, 880-883.
22. Min, L. et al, J. Am. Chem. Soc., 2019, 141, 15773-15778.
23. Motohashi, H. et al, Org. Lett. 2018, 20, 5340-5343.
24. Nguyen, A. L. et al, J. Org. Chem., 2018, 83, 3109-3118.
25. Rauch, M. et al, J. Am. Chem. Soc., 2019, 141, 17754-17762.
26. Schmidt, C. Nat. Biotechnol., 2017, 35, 493-494.
27. Sowalch, M. F. et al. Tetrahedron Lett., 2017, 58, 398-400.
28. Tokala, R. D. ot al, J. Org. Chem., 2019, 84, 5504-5513.
29. Xie, C. ot al, Org. Biomol. Chem., 2014, 12, 7836-7843.
30. Yang, Y. F. et al, Macromolecules, 2004, 37, 7918-7923.
31. Zhang, P. et al, Angow. Chem. Int. Ed. 2013, 52, 7869-7873.
32. Zhao, Y. et al, Theor. Chem. Acc., 2008, 120, 215-241.

1. A method for making a substituted 2,2-difluoroethanol, the method comprising contacting a pentafluoro-gem-diol of Formula I with molecular bromine, a carbonate base, and DMSO: 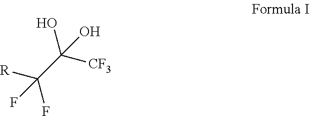
wherein R comprises a substituted or unsubstituted aryl or heteroaryl group having Formula II or a ketone having Formula III: 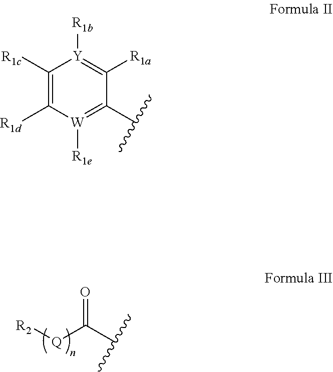
wherein, in Formula II, W and Y are independently C or N;
wherein R₁ₐ, R_{1c}, and R_{1d} are independently selected from hydrogen, nitro, halogen, cyano, trifluoromethyl, hydroxyl, linear or branched C₁-C₁₀ alkyl, or any combination thereof;
wherein, when Y is C, R_{1b} is selected from hydrogen, nitro, halogen, cyano, trifluoromethyl, hydroxyl, linear or branched C₁-C₁₀ alkyl, or any combination thereof; and
wherein, when W is C, R₁ₑ is selected from hydrogen, nitro, halogen, cyano, trifluoromethyl, hydroxyl, linear or branched C₁-C₁₀ alkyl, or any combination thereof;
wherein, in Formula III, n is from 0 to 10;
wherein when n is 1, Q is —CH₂—, and when n is from 2 to 10, Q is —CH₂— or —CH—; and
wherein R₂ is selected from a substituted or unsubstituted C₆-C₁₀ aryl or heteroaryl group, an adamantyl group, or any combination thereof.

2. The method of claim 1, wherein the pentafluoro-gem-diol of Formula I is selected from: 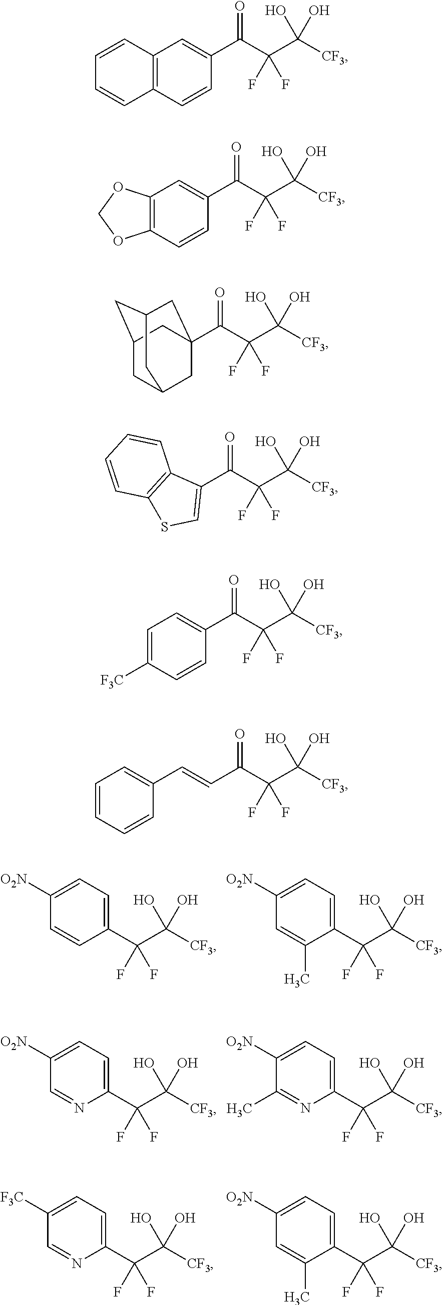
or any combination thereof.

3. The method of claim 1, wherein the carbonate base comprises Cs₂CO₃, Na₂CO₃, Li₂CO₃, or any combination thereof.

4. The method of claim 1, wherein the carbonate base is Cs₂O₃.

5. The method of claim 1, wherein the DMSO is DMSO-d₆.

6. The method of claim 1, further comprising contacting the pentafluoro-gem-diol with LiBr, molecular sieves, proton sponge, or any combination thereof.

7. The method of claim 1, wherein the method is performed at a temperature of from about 10° C. to about 60° C.

8. The method of claim 1, wherein the method is performed under argon.

9. The method of claim 1, wherein the substituted 2,2-difluoroethanol has Formula IV: 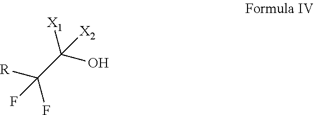
wherein X₁ and X₂ are H or D;
wherein R comprises a substituted or unsubstituted aryl or heteroaryl group having Formula II or a ketone having Formula III: 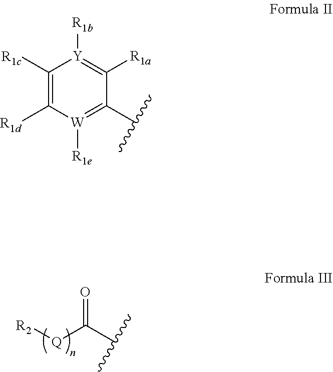
wherein, in Formula II, W and Y are independently C or N;
wherein R₁ₐ, R_{1c}, and R_{1d} are independently selected from hydrogen, nitro, halogen, cyano, trifluoromethyl, hydroxyl, linear or branched C₁-C₁₀ alkyl, or any combination thereof;
wherein, when Y is C, R_{1b} is selected from hydrogen, nitro, halogen, cyano, trifluoromethyl, hydroxyl, linear or branched C₁-C₁₀ alkyl, or any combination thereof; and
wherein, when W is C, R₁ₑ is selected from hydrogen, nitro, halogen, cyano, trifluoromethyl, hydroxyl, linear or branched C₁-C₁₀ alkyl, or any combination thereof;
wherein, in Formula III, n is from 0 to 10;
wherein when n is 1, Q is —CH₂—, and when n is from 2 to 10, Q is —CH₂— or —CH—; and
wherein R₂ is selected from a substituted or unsubstituted C₆-C₁₀ aryl or heteroaryl group, an adamantyl group, or any combination thereof.

10. The method of claim 9, wherein the substituted 2,2-difluoroethanol of Formula IV is selected from: 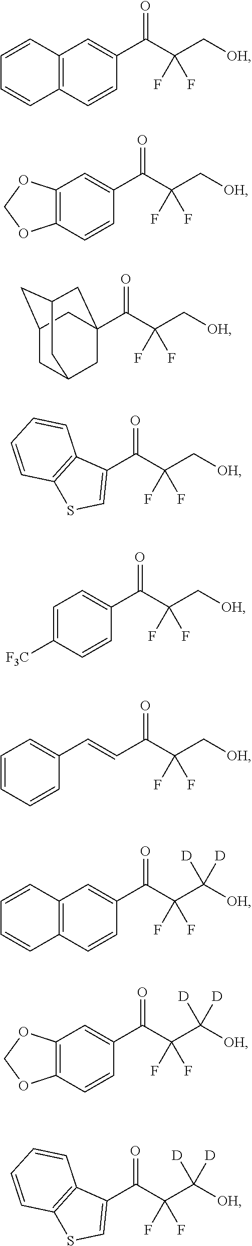 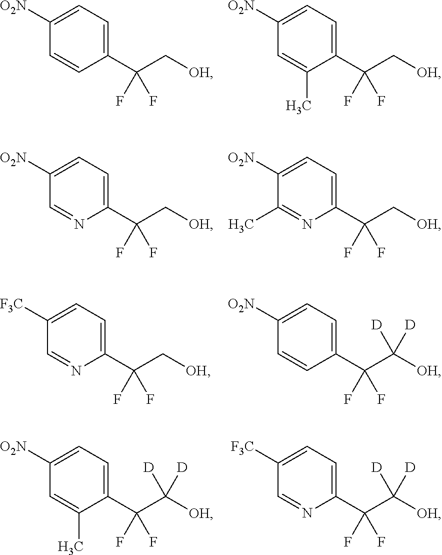
or any combination thereof.

11. A substituted 2,2-difluoroethanol having Formula IV: 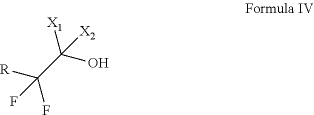
wherein X₁ and X₂ are H or D;
wherein R comprises a substituted or unsubstituted aryl or heteroaryl group having Formula II or a ketone having Formula III: 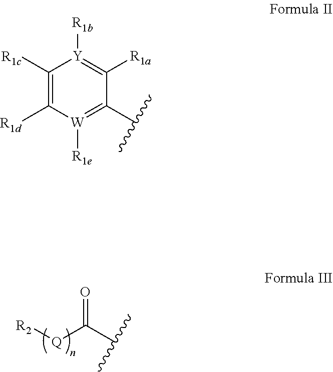
wherein, in Formula II, W and Y are independently C or N;
wherein R₁ₐ, R_{1c}, and R_{1d} are independently selected from hydrogen, nitro, halogen, cyano, trifluoromethyl, hydroxyl, linear or branched C₁-C₁₀ alkyl, or any combination thereof;
wherein, when Y is C, R_{1b} is selected from hydrogen, nitro, halogen, cyano, trifluoromethyl, hydroxyl, linear or branched C₁-C₁₀ alkyl, or any combination thereof; and
wherein, when W is C, R₁ₑ is selected from hydrogen, nitro, halogen, cyano, trifluoromethyl, hydroxyl, linear or branched C₁-C₁₀ alkyl, or any combination thereof;
wherein, in Formula III, n is from 0 to 10;
wherein when n is 1, Q is —CH₂—, and when n is from 2 to 10, Q is —CH₂— or —CH—; and
wherein R₂ is selected from a substituted or unsubstituted C₆-C₁₀ aryl or heteroaryl group, an adamantyl group, or any combination thereof.

12. The substituted 2,2-difluoroethanol of claim 11, wherein X₁ and X₂ are D.

13. The substituted 2,2-difluoroethanol of claim 11, wherein X₁ and X₂ are H.

14. The substituted 2,2-difluoroethanol of claim 11, wherein R is Formula III and Q is 0.

15. The substituted 2,2-difluoroethanol of claim 14, wherein R₂ is selected from 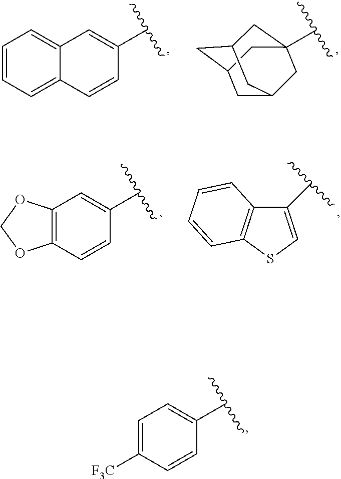 [SMILES: *c1ccc2ccccc2c1 ; *C12CC3CC(CC(C3)C1)C2 ; *c1ccc2c(c1)OCO2 ; *c1csc2ccccc12 ; *c1ccc(C(F)(F)F)cc1]
or any combination thereof.

16. The substituted 2,2-difluoroethanol of claim 11, wherein R is Formula II.

17. The substituted 2,2-difluoroethanol of claim 16, wherein W is N and Y is C.

18. The substituted 2,2-difluoroethanol of claim 16, wherein W and Y are both C.

19. The substituted 2,2-difluoroethanol of claim 11, wherein the substituted 2,2-difluoroethanol of Formula IV is selected from: 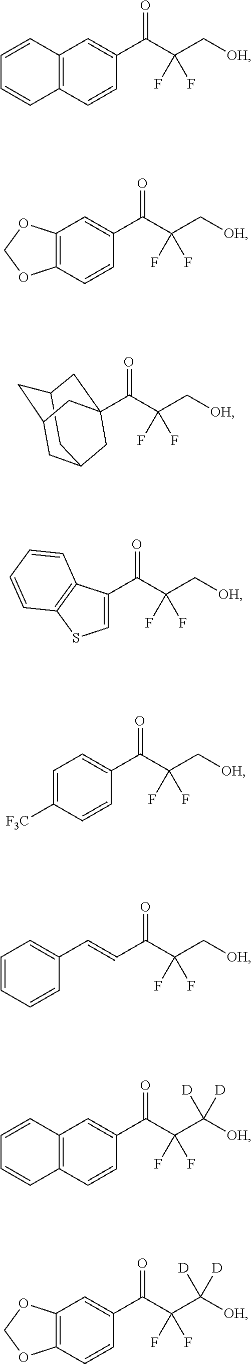 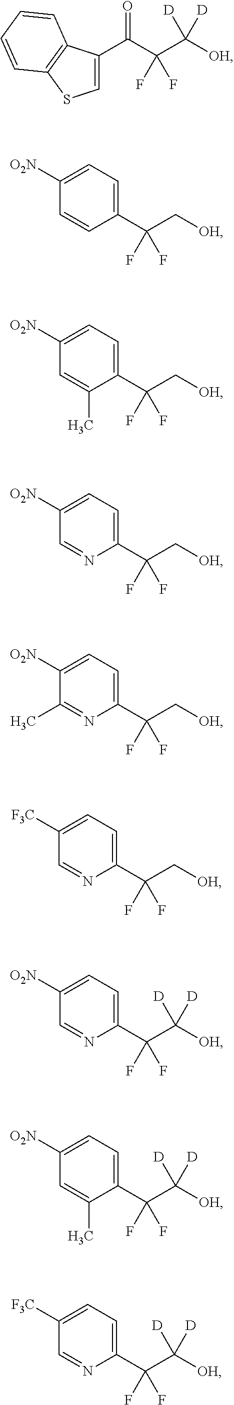
or any combination thereof.

20. A solvent or co-solvent for organic synthesis comprising the substituted 2,2-difluoroethanol of claim 11.
